(19) United States
(12) Patent Application Publication
(10) Pub. No.: US 2011/0010099 A1
(43) Pub. Date: Jan. 13, 2011

(54) Correlation Analysis of Biological Systems

(76) Inventors: Aram S Adourian, Woburn, MA (US); Matej Oresic, Espoo (FI); Doris Damian, Newton, MA (US); Eric K. Neumann, Lexington, MA (US); Thomas Plasterer, Marlborough, MA (US); Ezra Jennings, Cambridge, MA (US)

Correspondence Address: K&L Gates LLP, STATE STREET FINANCIAL CENTER, One Lincoln Street, BOSTON, MA, 02111-2950, US

(21) Appl. No.: 11/992,257

(22) PCT Filed: Sep. 19, 2006
(86) PCT No.: PCT/US06/36247

§ 371 Date: Sep. 10, 2010

Related U.S. Application Data
(60) Provisional application No. 60/718,561, filed on Sep. 19, 2005.

(51) Int. Cl. G06F 19/00; G01N 33/48

(52) U.S. Cl. 702/19

(57) ABSTRACT

The present teachings relate to gaining insight into biological states, e.g., disease states or drugged states, by gathering, integrating, and combining biomolecular data. More particularly, the present teachings relate to methods and systems for profiling a state of a biological system, finding accessible biomarkers representative of the state of a biological system, and deriving insights into the biochemistry of a biological system for therapeutic, diagnostic, prognostic and other purposes.

### FIELD

The present teachings relate to gaining insight into biological states, e.g., disease states or drugged states, by gathering, integrating, and combining biomolecular data. More particularly, the present teachings relate to methods and systems for profiling a state of a biological system, finding accessible biomarkers representative of the state of a biological system, and deriving insights into the biochemistry of a biological system for therapeutic, diagnostic, prognostic and other purposes.

### BACKGROUND

An important challenge in profiling biological states of mammals and in the development of new drugs for complex, multi-factorial diseases is the identification and validation of biomarkers. One definition of a biomarker is a measurable biochemical or set of biochemicals which reflect accurately the biological state of a system. Moreover, it appears that any single biomolecule has limited information content. Multiple biomolecules or patterns in the appearance and quantities of biomolecule, especially where multiple levels of the biological system are considered simultaneously, may be preferable as a means to characterize and diagnose homeostasis or disease states in an animal. One of the primary difficulties in biomarker discovery, selection and validation is that when a biological system is perturbed, for example by administration of a drug, a plethora of changes in analytes are detected. However, only a few of these changes may be specific to the perturbation, while many will be downstream consequences of the perturbation, and thus not specific to the perturbation. For example, administration of a toxic drug compound to an animal will result in many changes in its biological state, but only a subset of those changes will be specific to the toxicological insult and drug-target interaction. The majority of changes will be simply reflective of the general deterioration of the health of the animal. Furthermore, in many fields, e.g. drug discovery and development, health care, disease diagnosis and prognosis, drug side-effect diagnosis and prognosis, drug efficacy diagnosis and prognosis, and understanding disease etiology, it is important to obtain a biomarker which is both a true surrogate for the state of a biological system, and is readily accessible to the practitioner. Such biomarkers typically are found in body fluids such as blood, urine or other secretions or excretions of the organism.

The current strategy for the discovery of second generation candidate compounds, in a class of drugs designed to interact with a specific molecular target, is to seek ever more selective compounds for the target by differential in vitro screening of molecules in an array of available “on-target” and “off-target” assays. This approach usually produces a few improved follow-on drugs before the areas for additional improvement in drug performance based upon the efficacy and side effects of the drugs in patients are found to be unrelated to the drug properties measured in the screening assays. In parallel, or subsequently, a new target for drug discovery soon becomes fashionable and the “first-in-class followed by improved second-generation drugs” cycle repeats itself until disconnect is again reached between the effects of the second-generation drug candidates in patients and the early-stage screening assays. This situation arises because, beyond the primary and secondary outcome measures and a handful of conventional vital signs and clinical chemistries assessed in late-stage clinical trials, there is generally no useful information fed back from clinical trials to early-stage drug discovery to aid the process of designing improved drugs.

Accordingly, there is a need for methods and systems that facilitate analysis of a biological system as a whole, and that permit development of biomarkers truly characteristic of and directly related to the biology of a specific biological state. There also is a need for the development of new tools applicable broadly across the drug discovery and development process. Such methods and tools would advance the study of disease, and the discovery and development of pharmaceutical products.

### SUMMARY

Applicants are pioneers in a field known as “systems biology.” In contrast to analysis of an individual aspect of a biological system, systems biology is the study of biology as an integrated system including genetic, genomic, transcriptomic, proteomic and metabolomic components, and their pathways, which are in flux and interdependent. Rather than artificially simplifying the inherent complexity of biological processes that underlie the biology of a complex organism, e.g., the biological processes involved in human diseases or that govern drug responses, the methods and systems described herein embrace the complexities and interdependencies contained within all biological systems. By appropriately considering the complexity, a skilled artisan can undertake biological research at the systems level, developing cause and effect insights and profiles or biomarkers characteristic of a specific biological state of a specific biological.

Most studies in the life sciences and pharmaceutical drug discovery and development fields concentrate on analyzing data related to a change in the abundance of a measured analyte—be it a protein, gene transcript, metabolite or other—between or among pre-defined groups of samples, using univariate statistics such as the Student's t-test. What is less often considered is the statistical correlation structure among the measured analytes, which may be studied independently of pre-defined group designations. Indeed, it is often the case that in a comparison of two or more biological states, two or more measured analytes will be found to be very highly correlated due to, e.g., sharing of a common biological process by which they are biochemically co-regulated. However, none of these analytes considered individually would reveal a statistically significant fold-change between biological states, or serve as a reliable marker for the biological state. Such statistical or mathematical correlations among measured analytes can define and promote understanding of the state of a biological system, and suggest novel therapeutic, diagnostic and prognostic interventions by pharmaceutical agents for the modification or maintenance of the biological system. Thus, fold-change and correlation may be thought of as two complementary perspectives on data analysis for biological states.

Simply stated, the present teachings provide new ways of analyzing complex biochemical information from samples taken from organisms, such as human or animal subjects, and applying statistical and bioinformatic analyses to elucidate the correlation structure of this information. This enables development of accessible diagnostic or prognostic biomarkers truly characteristic of a biological state, selection of novel therapeutic targets for intervention, and probing biological systems in a new way.

It has now been discovered that a given biological state can be characterized by the pattern of correlations (multiple pairs, triads, or groups of data points whose levels correlate) among biomolecules in a sample taken from an individual in the biological state. Thus, a given biological state of an animal can be determined by analyzing (i.e., measuring relative amounts of) a multiplicity of biomolecules (e.g., genes, gene transcripts, lipids, proteins, and/or metabolites—frequently tens to hundreds of such biomolecules) present in one or more samples from the animal, conditioning and examining the data in a standardized way so as to determine whether certain of them correlate to one another either positively or negatively, optionally producing some form of correlation map, and then comparing the correlations found in the test sample to a reference set of correlations. The test animal will be in the same biological state as the animal(s) that produced the reference sample. Thus, the present teachings provide insight into a biological state at a systems level so that connections, correlations, and relationships among thousands of diverse, measurable molecular components can be achieved.

Thus, for example, in a given sample, if data points:
A, B and C all increase together;
F, H and K all decrease together;
when J increases, X and L decrease; and
when S decreases, U, I and O increase,
   then this means that the sample is from a test subject in a particular biological state (e.g., has a type of diabetes, is in some specific toxic state, etc) and not in some other state. This exemplary correlation pattern indicates that the subject is in the biological state because this pattern of correlation previously had been demonstrated to be characteristic of the biological state as indicated by parallel analysis of a the study set.

In another aspect, the present teachings permit correlation analysis across compartments within an individual. Thus, the rise and fall of the levels of biomolecules in an organ or tissue, which is characteristic of that organ or tissue being in a particular biological state, can be correlated to the rise and fall of biomolecules in an accessible body fluid such as blood or urine. This permits the researcher to develop sets of biomarkers in, e.g., serum, that are directly correlated biochemical changes in an organ or tissue, without the necessity of biopsy and direct tissue analysis. This is a significant improvement over previously developed empirical methods of profile development based on fold changes in concentrations.

Furthermore, the correlation analysis can lead to the discovery of biomolecules that exhibit a high clustering coefficient, meaning that, when a test animal is in a particular biological state, the level of the biomolecule correlates positively or negatively with multiple other biomolecules. Such high clustering coefficient biomolecule may be pivotal in the biological state under study (e.g., disease) and it may be that inhibitors of the biomolecule's function, or agonists or antagonists of the biomolecule may be effective in the treatment of the disease or in mitigation of its symptoms.

A reference set of correlations can be made by study of a group of test animals, e.g., experimental animals or human volunteers, confirmed to be in a biological state of interest (or by multiple measurements on one or a smaller group of test animals over time during the development of disease, after receiving different drug dosages, after receiving different drugs with similar mechanisms of action, or from different biological compartments). For example, 50 test subjects may be sampled. The relative amounts of a relatively large group of biomolecules are examined to determine their relative or absolute concentrations. For example, spectrometric data may be collected using any one of a large group of analytical instruments, many of which are commercially available, or by any appropriate known technique, e.g., mass spectrometry, liquid chromatography, gas chromatography, array hybridization, or nuclear magnetic resonance spectroscopy, various combinations thereof, or techniques hereafter developed. The data are conditioned (e.g. normalized to be made comparable or validated by other statistical techniques) to produce data points. Data points from animals within the test group are inspected for similarity (or, in the terms of the statistician, ‘concordance’, ‘coherence’, ‘coincidence’, ‘interdependence’, ‘association’, ‘co-ordinate’, ‘attendant’, ‘concurrence’, ‘isochronicity’, or ‘synchronicity’) in the measured amounts of sets of biomolecules, e.g. pairs or triads, etc., of biomolecules. For example, between each selected pair of data points in the test group, a +1 may be assigned for a positive correlation, a −1 for negative correlation, and 0 for no correlation. As such, the data are reduced to a set of correlation coefficients between or among measured biomolecules ranging from −1 to +1. One may and typically does take into account the strength of the correlations, focusing on values, e.g., r<−0.75 or r>+0.75. Some or all of the negative and/or positive correlations may be used as components of a “biomarker” or “profile” that characterizes the biological state, i.e., to produce a data set that if reproduced by analysis in a new individual indicates that that individual is in the biological state.

Data points from animals within a control group may also be inspected in the same manner as above, and the resulting control correlation data set compared for similarities and/or differences with test groups, thereby to improve the acuity or precision of the correlation map or data set, by validating selected correlated data as being characteristic of the biological state under study or by suggesting removal of points that do not serve to distinguish an animal in the biological state under study from controls.

The data set may reside in the memory of a computer. Conveniently, the data set may be translated into a visual format, i.e., used to produce a correlation map having a visual appearance indicative of the biological state under study. Correlation maps permit a researcher or clinician to assess by visual inspection whether a given individual is or is not in the biological state. The correlation map may take many specific forms, as discussed herein.

The present teachings provide methods and systems to analyze complex clinical samples of organisms including humans at a systems biology level to provide new information about the state of a biological system that was previously unobtainable through traditional chemistries, genomic studies, or biological data analysis techniques alone. Using the methods and systems described herein, it is possible to gain insight into biological pathways and mechanisms of disease and drug response. These methods and systems can analyze and integrate data at the biomolecular component type level to create knowledge that advances pharmaceutical research and development by providing new insights into the molecular mechanisms of health and disease, and to promote the development and discovery of novel therapeutics to treat disease.

Such knowledge then may be used directly for the development of therapeutic agents or biomarkers, may be used in combination with clinical information, and/or may serve as a basis for directed, hypothesis-driven experiments designed to further elucidate biochemical pathways and pathophysiologic mechanisms. Further, tracking changes of a profile of a biological system can improve many aspects of pharmaceutical discovery and development, including drug safety and efficacy and drug response, and can elucidate the etiology of disease.

Within the framework described above, an enormous number of practical, medically-relevant uses of the technology emerge.

One high value use for correlation data sets or maps is in pharmacology studies. As an example, data sets of diseased and healthy individuals can be constructed. A drug candidate then is administered to a diseased individual, and a data set is generated from a sample taken from the individual while under the influence of the drug. This can be compared to the data set of one or more healthy individuals, a diseased individual treated successfully with a different drug, or the data set of a diseased individual. Comparison of the data can suggest that the drug candidate might be efficacious, as it might have altered the pattern toward the healthy data set, or altered the pattern toward the pattern of the successfully drugged individual. Any drug candidate can be assessed in this manner, including, in particular, known drug substances for which new uses are proposed, new compound which was discovered empirically or designed using a rational drug design method aimed at the disease state, and combinations of drugs in which neither, one, or both are known to be efficacious in treating the disease.

Another important use of the present teachings is in assessing toxicity of a substance or combination of substances, such as a drug candidate. In this embodiment, the drug is administered to a test mammal, such as a human subject or experimental animal, and a correlation map or pattern is generated from a sample taken from the subject. The test correlation pattern is then compared to one or more reference patterns (data set). These are generated, for example, from one or more samples from a mammal of the same species to which a known substance toxic to the mammal has been administered, from the same individual mammal before the substance has been administered, from several mammals exhibiting a variety of different toxic responses, or from a mammal administered the substance which is known to tolerate the substance. If, for example, the test correlation pattern resembles the toxic reference pattern, but not the pattern generated from non-drugged healthy mammals, that may be an indicator of the possible toxicity of the drug candidate to the test animal. The comparisons to determine toxicity (as is the case with other determinations according to the present teachings), typically is done with the aid of a computer, in which case no map or visual image need be generated. Alternatively, the data can be processed to form one or more correlation maps or displays, which can be visually compared by a physician or a pharmaceutical research scientist.

Correlation data sets and maps also can be used in studies in which patients are grouped, in advance of the correlation analysis, into one which has been observed to respond in one phenotypic manner to a drug, e.g., exhibits a mitigation of the disease, and another which exhibits a different phenotypic response, e.g., no mitigation. Upon comparison of maps produced as disclosed herein from data generated from samples taken from each group, clues to the biochemical basis of the observed phenotypic differences appear as characteristic associations of biomolecules. These insights also may permit the researcher to predict, by analysis of a sample from a candidate for the drug, in advance of drug administration, or after administration of a micro-dose of a drug, who will benefit from the drug and who will not.

Correlation analysis data and maps also can be used to signal possible side effects of a drug, induced either by a candidate drug to be administered to a human or animal, or induced by an established drug only in a subgroup of patients. To detect possible side effects, a map generated from a sample from a test subject to whom the drug has been administered is compared to a reference map generated from informative samples, e.g., samples from subjects that have been administered the same or a different known drug which in them caused side effects, and/or from subjects to whom drugs have not been administered. This use of the technology finds particular utility in clinical trials, where a potentially useful drug might have side effects in a small portion of the population which is not easily identifiable by conventional techniques. If an individual being considered for enrollment in a trial provides a sample which generates a map which closely resembles reference maps characteristic of side effects for the class of drugs in which the drug candidate belongs, that subject is excluded from the trial. Similarly, individuals can be tested, and their maps compared to reference maps to identify patients who are likely to suffer side effects from treatment with the drug, are likely to benefit, or are unlikely to benefit.

“Systems pharmacology” can enable dramatic improvements upon marketed drugs of a structural or mechanistic class by establishing a role for correlation analysis data and maps as the system-wide activity measure for chemical structure-activity studies. Features of the correlation analysis data sets obtained from studies in patients with marketed drugs or late-stage drug candidates can be correlated with efficacy and side-effect measures in the same patients. If the features of the correlation analysis data sets obtained in patients can also be identified in the best animal model, irrespective of whether the relationship of those features to the disease or drug response can be understood, then drug hunters will use animal model correlation analysis data sets that reflect human efficacy and safety as criteria for selecting the next generation of development candidates. Such, comparative reverse systems pharmacology would constitute the first total quality improvement clinical-to-discovery feedback program in the pharmaceutical value chain, and a radical departure from current drug improvement practices.

Combination drug therapy has undergone several stages of acceptance and utility in the past, from undesirable through acceptable from a compliance perspective to an innovative activity. An appreciation of the system-wide nature of diseases and an insight into the regulation of homeostasis via multiple biochemical mechanisms and multi-compartment interactions could unlock the potential for a totally new perspective on the discovery of combination drug products. For example, many of the drug candidates that have failed in clinical development on the basis of limited efficacy, despite clear evidence that their targets play some role in a particular disease mechanism, could be revived in combination with marketed drugs or other failed drug candidates. Similar revival opportunities exist for compounds that have failed because safety issues were revealed at the efficacious doses, because as components of combination drug products it might be possible to administer those compounds at doses below the threshold at which the safety issues arose. Correlation analysis data sets and maps can play a significant role in the development of such techniques as they permit development of true surrogates of biological states and a reliable means to assess a subject accurately at a cogent, systems biology level.

Thus in various aspects, all as more particularly pointed out in the appended claims, the present teachings provide correlation analysis data sets that effectively serve as biomarkers for a given biological state, which are embodied as a table or other tangible form, or be stored as a set of values in the memory of a computer or on a data storage medium. The present teachings also provide methods for using the data sets and the clustering coefficients which can emerge from a correlation analysis to help identify possible new targets addressable by a drug molecule for therapeutic, prophylactic or analgesic use. The present teachings also provide methods of assessing drug efficacy using the data sets; technique useful in systems biology analysis broadly; methods of assessing toxicity of a drug candidate or other substance; clinical diagnostic methods; various species of patient segmentation protocols, including micro-dosing techniques, useful in the practice of personalized medicine or selection of patients in clinical trials; and methods for determining the mechanism of action of drugs, e.g., whether two or more drug candidates intended for treatment of the same or related diseases operate by the same or a different pathway.

Other aspects, advantages and features of the present teachings will become apparent from the following claims, and from the figures and detailed description, which illustrate the principles of the present teachings by way of example only.

### BRIEF DESCRIPTION OF THE DRAWINGS

The foregoing and other objects, features, and advantages of the present teachings will be more fully understood from the following description of various illustrative embodiments, when read together with the accompanying drawings. The drawings are not intended to limit the scope of the present teachings in any way.

FIG. 1 is a graphical representation of a correlation network.

FIG. 2 depicts an example of a correlation demonstrating a positive correlation across 20 animals between two features from a plasma GC-MS platform and a peptide feature from a LC-MS proteomics platform.

FIG. 3 depicts an example of a correlation demonstrating a negative correlation across 20 animals between two features from a plasma LC-MS platform and a peptide feature from a LC-MS proteomics platform.

FIG. 4 depicts another example of a correlation demonstrating a positive correlation across 9 animals between two features, one from a serum high density lipoprotein measurement platform and one from an adipose tissue messenger RNA feature from a transcriptomics platform.

FIG. 5 depicts another example of a correlation demonstrating a negative correlation across 9 animals between two features, one from a serum high density lipoprotein measurement platform and one from an adipose tissue LC-MS lipid platform.

FIG. 6 depicts an example of a correlation demonstrating a correlation near zero across 9 animals between two features, one from a serum high density lipoprotein measurement platform and one from an adipose tissue messenger RNA feature from a transcriptomics platform.

FIG. 7 depicts a correlation convolved with state-specific group effects and the correlation deconvolved from such effects.

FIG. 8 depicts the results of a jack-knifing cross-validation routine to guard against outlier-driven correlations.

FIGS. 9a-9k are flow charts illustrating process steps that can be used in the practice of the present teachings.

FIG. 10 depicts histograms of differences in about 1000 measured features across 2 samples.

FIG. 11 depicts coefficients of variance as determined from samples for 8 measurements from an LC-MS analytical platform before data normalization (solid lines) and after data normalization (dashed lines).

FIG. 12 depicts a correlation network in liver tissue, with all measured analytes as nodes, in the DV biological state.

FIGS. 13-15 depict subsets of a larger correlation network of the type exemplified in FIG. 12.

FIG. 16 depicts scatter plots of the relative abundance levels of two selected nodes and the corresponding edge from the correlation networks of FIGS. 14 and 15.

FIGS. 17-20 are graphical representations of correlation networks centered around node “A.”

FIG. 21 depicts a set of nodes and edges chosen from a larger correlation network (e.g., as exemplified in FIG. 12), and also shows the results of a gene ontology categorization (dashed lines) of a subset of the nodes.

FIG. 22 depicts a cross-tissue correlation network.

FIG. 23 depicts the correlation network of FIG. 22 filtered to produce a smaller correlation network focusing on 3 serum analytes and the tissue analytes to which they correlate.

FIG. 24 depicts a set of nodes and edges beginning with the correlation network of FIG. 23 and supplemented by mapping analytes in FIG. 23 to the Gene Ontology Biological Process hierarchy.

FIG. 25 depicts a correlation sub-network.

FIG. 26 depicts a biochemical cycle in which both an enzyme and a metabolite are known to play a role.

FIG. 27 depicts a correlation matrix centered on the hepatic Enzyme X, illustrating correlations both to other liver analytes and analytes in plasma.

FIG. 28 depicts a screen shot of Seer™, which can be used to visualize correlation networks.

FIG. 29 depicts box plots of the distribution of two analytes, 157.4208 and 185.421, which show significant differential expression in Group 3 vs. Group 1 comparison.

FIG. 30 depicts box plots of the distribution of two analytes, 577.0975 and 844.0926, which show significant differential expression in the Group 3 vs. Group 1 comparison.

### DETAILED DESCRIPTION

### Systems Biology Studies

### Introduction, Principles, and Definitions

The methods and systems disclosed herein rely on measurements of constituents of biological samples, including metabolites, proteins, genes, gene transcripts, lipids sugars, etc. to permit a skilled artisan to understand a biological system more holistically and in greater depth than an approach that examines only one or a subset of these factors. Understanding the biological system as a whole can improve multiple aspects of pharmaceutical discovery and development, including drug safety and efficacy, drug response, and the etiology of disease. A systems biology platform integrates genomics, transcriptomics, proteomics, metabolomics, and bioinformatics, and results in data integration and knowledge management platform that generates connections, correlations, and relationships among thousands of measurable molecular components to better understand and to develop of a profile of a state of a biological system. Resulting profiles can be combined with clinical information to increase the knowledge of a state of a biological system.

A “profile” of a biological system is a summary or analysis of data representing distinctive features or characteristics of a biological state in a biological system, e.g., in an animal, e.g., a mammal such as a human, or in some compartment of an animal, such as liver, heart, or CNS. The data can include measurements or features (e.g. concentrations or absolute values) relating to various biological sample types (e.g., blood serum and saliva), types of measurement techniques (e.g., mass spectrometry (MS) and nuclear magnetic resonance spectrometry (NMR)), and biomolecular component types (e.g. metabolites and transcripts). The data can further include univariate or multivariate statistics on changes in abundance of one or more measurements or features between or among a priori defined groups of samples, or univariate or multivariate statistics on the statistical correlation structure among measurements or features. The data often are spectral or chromatographic features that are in the form of a graph, table, or some similar data compilation. A profile typically is a set of data features that permit characterization of a state of a biological system. A profile can also be embodied as a tabular or graphical representation of the correlations or relationships between and among measurements or features that permits characterization of a state of a biological system. Such a profile often is termed a “biomarker,” although it comprises a compilation of data relating to many individual biomolecules.

Thus, a profile includes data relating to plural individual biomolecules, individual ones of which often previously have been referred to as “biomarkers,” in the sense that their presence or level in a sample suggested that the sample was from a subject in a particular biological state. “Biomolecule” refers to the molecules found in a living system, and may be of various known biological component types. Thus, a profile can be considered to be a set of data, e.g., spectral or chromatographic features, derived from measurement of selected biomolecules that collectively permit characterization of a state of a biological system. A profile also can be considered to include correlations and other results of analyses of the data sets. The correlation data sets and maps of the present teachings comprise one form of profile.

A “state of a biological system” refers to a condition in which the biological system exists, either naturally or after a perturbation. Any biological state or phenotype may be examined using the processes of the present teachings. Non limiting examples include a normal, healthy state when an animal is in homeostasis often used as a control), a diseased state, a toxic state, or an aged state. Particular biological states are induced by factors internal and external to the animal, such as by biochemical regulation (e.g., apoptosis), ageing, an environmental stimulus, or mental or physical stress or deprivation. The biological state may be a pathologic, diseased, well, toxic, homeostatic, hunger induced, environmentally induced, exercise induced, drug induced, placebo induced, or mental illness induced.

Development of a profile of a biological state permits comparison of one profile to another to determine whether two subjects are in the same or a different biological state, e.g., healthy or suffering from a particular disease. A biological system is better characterized using a multivariate analysis rather than using multiple measurements of the same variable because multivariate analysis envisions the biological system as a whole. Disparate data from multiple, different sources is treated as if in a single dimension rather than in multiple dimensions. Consequently, the analysis of data is more informative and typically provides a profile that is more robust and predictive than one that is developed by systematically evaluating multiple components individually or one that relies on one particular biomolecular component type.

Prior art techniques for developing such profiles have been empirical, and based on fold changes in abundance of biomolecules. Thus, previously described techniques involve the examination of data relating to the concentrations of each of a groups of individual biomolecules found in a test group of individuals known to be in some biological state, and data obtained and treated in the same way from control individuals. When these data are compared, data features from groups of biomolecules that are found in the test, but not the control individuals emerge, and these are proposed as a biomarker.

A “biomolecular component type” refers to a class of biomolecules associated with biological systems. Genes and gene transcripts (which may be interchangeably referred to herein) are examples of biomolecular component types that generally are associated with gene expression in a biological system, and where the level of the biological system is referred to as genomic or functional genomic level. Proteins and their constituent peptides (which may be interchangeably referred to herein), are another example, associated with protein expression and modification, where the study of the biological system is referred to as proteomics. Glycoproteins and glycopeptides also are considered a biomolecular component type. Another example of a biomolecular component type is metabolites (which also may be referred to as small molecules), which generally are associated with the study of a biological system referred to as metabolomics. Metabolites include, but are not limited to, lipids, steroids, amino acids, organic acids, bile acids, eicosanoids, neuropeptides, vitamins, neurotransmitters, carbohydrates, ionic organics, nucleotides, inorganics, xenobiotics, peptides, trace elements, and pharmacophore and drug breakdown products.

The methods described herein may be used to develop a profile of a state of a biological system based on any single biomolecular component type as well as based on two or more biomolecular component types. Profiles comprising data from particular biomolecular component types facilitate characterization and understanding of different levels of a biological system. Thus systems biology studies can provide genomic profiles, transcriptomic profiles, proteomic profiles and metabolomic profiles, and permit their comparison, integration, and analysis. These methods may be used to analyze holistically measurements derived from one or more biological sample type, one or more type of measurement technique, or a combination of at least one each of a biological sample type and a measurement technique so as to permit the evaluation of similarities, differences, and/or correlations in a single biomolecular component type or across two or more biomolecular component types.

A “biological sample type” includes, but is not limited to, blood, blood plasma, blood serum, cerebrospinal fluid, bile acid, saliva, synovial fluid, pleural fluid, pericardial fluid, peritoneal fluid, sweat, feces, nasal fluid, ocular fluid, intracellular fluid, intercellular fluid, lymph urine, tissue, liver cells, epithelial cells, endothelial cells, kidney cells, prostate cells, blood cells, lung cells, brain cells, adipose cells, tumor cells, and mammary cells. The sources of biological sample types may be different subjects, the same subject at different times, and other permutations. Further, a biological sample type may be treated differently prior to evaluation such as using different work-up protocols.

A “measurement technique” refers to any analytical technique that generates or provides data that is useful in the analysis of a state of a biological system. For example, measurement techniques include, but are not limited to, mass spectrometry (“MS”), nuclear magnetic resonance spectroscopy (“NMR”), liquid chromatography (“LC”), gas-chromatography (“GC”), high performance liquid chromatography (“HPLC”), capillary electrophoresis (“CE”), gel electrophoresis (“GE”) and any known form of hyphenated mass spectrometry in low or high resolution mode, such as LC-MS, GC-MS, CE-MS, MS-MS, MSⁿ, and other variants. Measurement techniques include biological imaging such as magnetic resonance imagery (“MRI”), video signals, and an array of fluorescence, e.g., light intensity and/or color from points in space, and other high throughput or highly parallel data collection techniques.

Measurement techniques also include optical spectroscopy, digital imagery, oligonucleotide array hybridization, protein array hybridization, DNA hybridization arrays (“gene chips”), immunohistochemical analysis, polymerase chain reaction, nucleic acid hybridization, electrocardiography, computed axial tomography, positron emission tomography, and subjective analyses such as found in text-base clinical data reports. For a particular analysis, different measurement techniques may include different instrument configurations or settings relating to the same measurement technique.

A “measurement” refers to a value in a data set that is generated by or derived from a measurement technique. A “data set” includes measurements derived from one or more sources. For example, a data set can be a series of measurements collected by the same technique, i.e., a collection or set of data of related measurements. Further, data sets more broadly may represent collections of diverse data, e.g., protein expression data, gene expression data, metabolite concentration data, magnetic resonance imaging data, electrocardiogram data, genotype data, single nucleotide polymorphism data, and other biological data. That is, any measurable or quantifiable aspect of a biological system being studied may serve as the basis for generating a given data set.

A “feature” of a data set refers to a particular measurement associated with a data set relating to a measurement of a biomolecules, or relationship(s) between measurements of two or more of biomolecules. For example, as noted above, a profile typically is a set of data features that permit characterization of a state of a biological system.

“Data sets” may refer to substantially all or a sub-set of the data associated with one or more measurement techniques. For example, the data associated with the spectrometric measurements of different sample sources may be grouped into different data sets. As a result, a first data set may refer to experimental group sample measurements and a second data set may refer to control group sample measurements. In addition, data sets may refer to data grouped based on any other classification considered relevant. For example, data associated with the spectrometric measurements of a single sample source may be grouped into different data sets based on the instrument used to perform the measurement, the time a sample was taken, the appearance of a sample, or other identifiable variables and characteristics. In one important embodiment of the present teachings, as discussed herein, a data set is obtained from an accessible body fluid such as serum, urine or CSF and from tissue sampled from an organ of the same individual, or pairs of such samples, and the data sets they produce are obtained from plural individuals exhibiting the same biological state. One data set may include a sub-set of another data set. In addition, it should be realized that the term “data set” includes raw spectrometric data, data that has been preprocessed, e.g., to remove noise, to correct a baseline, to smooth the data, to detect peaks, and/or to normalize the data, and collections of data features that have been discovered to correlate.

“Spectrometric data” refers to any data that may be represented in the form of a graph, table, vector, array or some similar data compilation, and may include data from any spectrometric or chromatographic technique. The term “spectrometric measurement” includes measurements made by any spectrometric or chromatographic technique.

Central to the methods disclosed herein is the statistical analysis of a plurality of data sets. “Statistical analysis” includes parametric analysis, non-parametric analysis, univariate analysis, multivariate analysis, linear analysis, non-linear analysis, and other statistical methods known to those skilled in the art. Multivariate analysis, which determines patterns in apparently chaotic data, includes, but is not limited to, principal component analysis (“PCA”), discriminant analysis (“DA”), PCA-DA, canonical correlation (“CC”), cluster analysis, partial least squares (“PLS”), predictive linear discriminant analysis (“PLDA”), neural networks, and pattern recognition techniques. Also central to the methods disclosed herein is the statistical analysis of correlations among measurements. “Correlation analysis” includes parametric analysis, non-parametric analysis, linear and non-linear correlation, Pearson's correlation analysis, Pearson's Product Moment Correlation analysis, Spearman rank correlation analysis, Kendall correlation analysis, partial correlation, and other statistical correlation methods known to those skilled in the art.

A “correlation network” refers to any graphical representation of the correlation structure among a single or plurality of data sets (such as found in Oresic et al., “Phenotype characterization using integrated gene transcript, protein and metabolite profiling,” Applied Bioinformatics, 3(4):205-17 (2004)).

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously. Data from a given experiment may be used in a future experiment, and used retrospectively any number of times.

### Correlation Data Sets

As described and demonstrated herein, the data, measurements, and values used in the methods of the present teachings can be derived from a variety of different sources using a variety of different techniques. The data and values can be representative of different chemical entities as well as other quantitatively and/or qualitatively measurable and/or definable features or characteristics of a biological system. See, for example, U.S. Patent Application Publication Nos. US 2003/0134304 A1 and US 2005/0170372 A1; and International Publication Nos. WO 03/017177 A2 and 2005/020125 A2, the entire disclosures of which are incorporated by reference herein for all purposes.

It should be understood that the data, e.g., measurements and values, used in the present teachings are not just any numbers or qualitative information, but typically are obtained or derived from a sample of a biological system using a variety of techniques known in the art. That is, although the present teachings do not focus on the acquisition of the data, the methods of the present teachings often utilize data that had been measured, e.g., spectrometric measurements, whether directly as part of the present teachings or indirectly for some unrelated analysis that can be reported in the scientific literature or otherwise publicly available.

In various embodiments, the methods of the present teachings generally include evaluating with a statistical analysis a plurality of data sets of a biological systems and comparing features among the data sets to determine one or more sets of differences among at least a portion of the data sets so as to develop a profile for a state of a biological system based on the comparison. Typically, the data sets are preprocessed and evaluated using multivariate analysis. In some embodiments, more than one statistical analysis is performed on the plurality of data sets, on various permutations of the plurality of data sets, and/or on the results of a particular statistical analysis. For example, a profile may be developed by conducting separate correlation analyses on a plurality of data sets related to proteins and a plurality of data sets related to metabolites, then evaluating with statistical analysis the results of the individual analyses to develop a profile for the biological state of the system that includes both proteins and metabolites. Alternatively, the plurality of data sets relating to proteins and metabolites of the biological systems may be evaluated simultaneously.

In some embodiments, the analysis method comprises selecting a biological sample; preparing the biological sample based on the biochemical components to be investigated and the spectrometric techniques to be employed; measuring the components, for example, the high concentration components, in the samples using spectrometric and chromatographic techniques; measuring selected molecule subclasses using, for example, NMR and/or MS approaches; preprocessing the raw data; using statistical analysis, which will be described in more detail below, to analyze the preprocessed data to identify patterns in measurements; and using statistical analysis to combine data sets from distinct experiments and identify data patterns of interest. As disclosed herein, the elucidated data patterns of the present teachings usually are based on correlation analysis.

Thus the present teachings provide techniques for determining associations/correlations within, between, and among biomolecular component types of suitable data sets using linear, non-linear or other mathematical tools. In some embodiments, the methods and systems described herein involves using these associations and/or correlations to postulate networks of interacting biomolecular components to determine causality among these associations, and to establish hypotheses about the biological processes underlying the observations which give rise to the data sets.

Of course before performing statistical analysis, the raw data may and typically will be preprocessed to assist in the comparison of different data sets. In particular, to compare data across different biomolecular component types, appropriate preprocessing should be performed. Preprocessing of the data may include (i) aligning data points between data sets, e.g., using partial linear fit techniques to align peaks of spectra of different samples; (ii) normalizing the data of the data sets, e.g., using standards in each measurement to adjust peak height; (iii) reducing the noise and/or detecting peaks, e.g., setting a threshold level for peaks so as to discern the actual presence of a species from potential baseline noise; and/or (iv) other data processing techniques known in the art. Data preprocessing can include entropy-based peak detection as disclosed in U.S. Pat. No. 6,743,364, and partial linear fit techniques (such as found in J. T. W. E. Vogels et al., “Partial Linear Fit: A New NMR Spectroscopy Processing Tool for Pattern Recognition Applications,” Journal of Chemometrics, vol. 10, pp. 425-38 (1996)). Further, data may be processed by a variety of transformations including logarithmic transformation of measurement values, rank transformation of measurement values, scaling of measurement values to unit variance, mean-centering of measurement values, and other data transformation methods known to those skilled in the art.

The methods of the present teachings can include displaying all or a portion of the data, measurements, values, correlations and networks, and any other useful information that can be visualized. Such displaying can be helpful to discern patterns in the data and to assist in the interpretation of the results, e.g., a correlation network. However, it should be understood that not in all embodiments is displaying of data an essential feature as certain embodiments can provide technical character in an alternative way.

The present teachings also provide an article of manufacture where the functionality of a method disclosed herein is embedded on a computer-readable medium such as, but not limited to, a floppy disk, a hard disk, an optical disk, a magnetic tape, a PROM, an EPROM, CD-ROM, or DVD-ROM. The functionality of the method may be embedded on the computer-readable medium in any number of computer-readable instructions or languages such as FORTRAN, PASCAL, C, C++, BASIC and assembly language. Further, the computer-readable instructions may be written in a script, macro, or functionally embedded in commercially available software such as EXCEL or VISUAL BASIC. In other aspects, the present teachings provide systems adapted to practice the methods described herein.

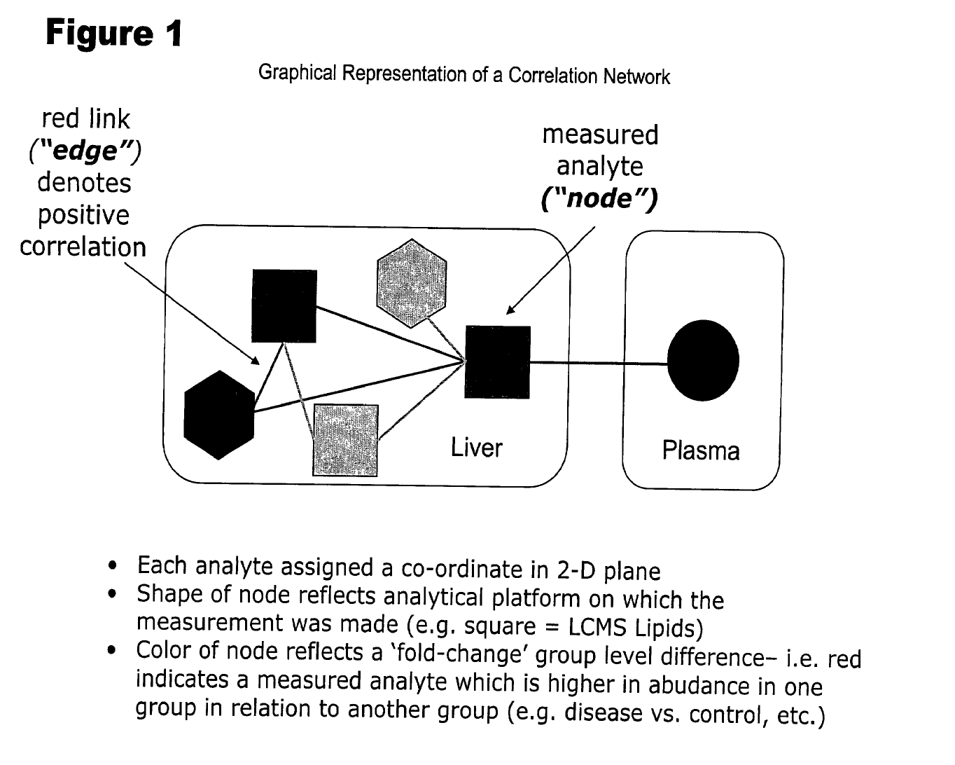

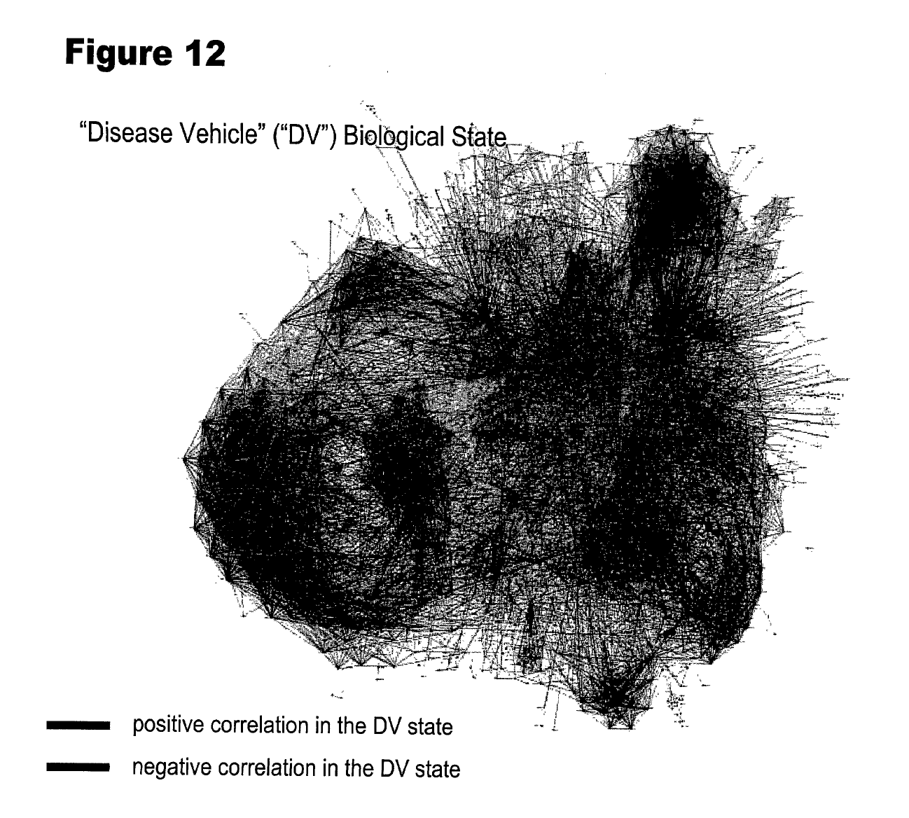

FIG. 1 shows a simple example of a graphical representation of a correlation network. As illustrated in this case, the correlation networks is displayed as a graphical representation of sets of pair-wise mathematical correlations between intensity values of measured features. Measured features are represented by ‘nodes’, and correlations between pairs of analytes are represented by links, or ‘edges’, which connect the corresponding nodes. Graph edges represent the pair-wise relationships between nodes. Each node is assigned a co-ordinate in the two-dimensional plane, such that the pair wise distances approximately reflect the similarity given by the correlation matrix; an edge is drawn between two nodes if their correlation exceeds a given quantitative threshold. Correlations can be derived for pairs of features measured either within or across tissues or biological compartments. Examples of such correlation graphs or networks are shown in FIGS. 1, 2, 3, 4, 5, and 6; more complex depictions of correlation networks are shown in FIGS. 12-15. There are many alternate graphical representations of correlation networks, limited only by the ingenuity and imagination of the scientist.

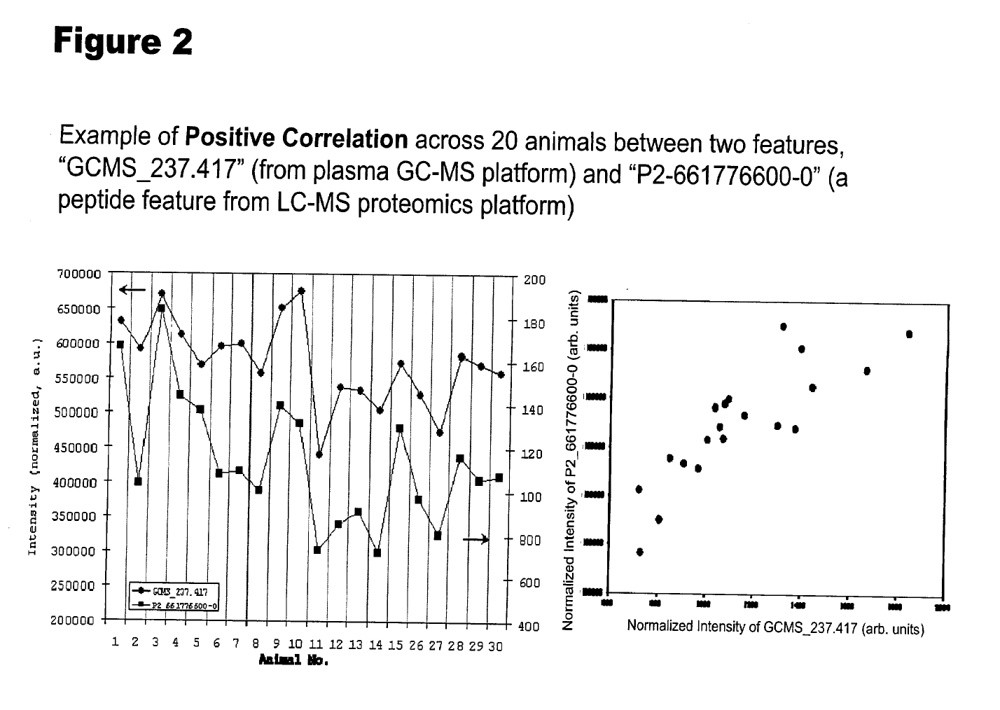

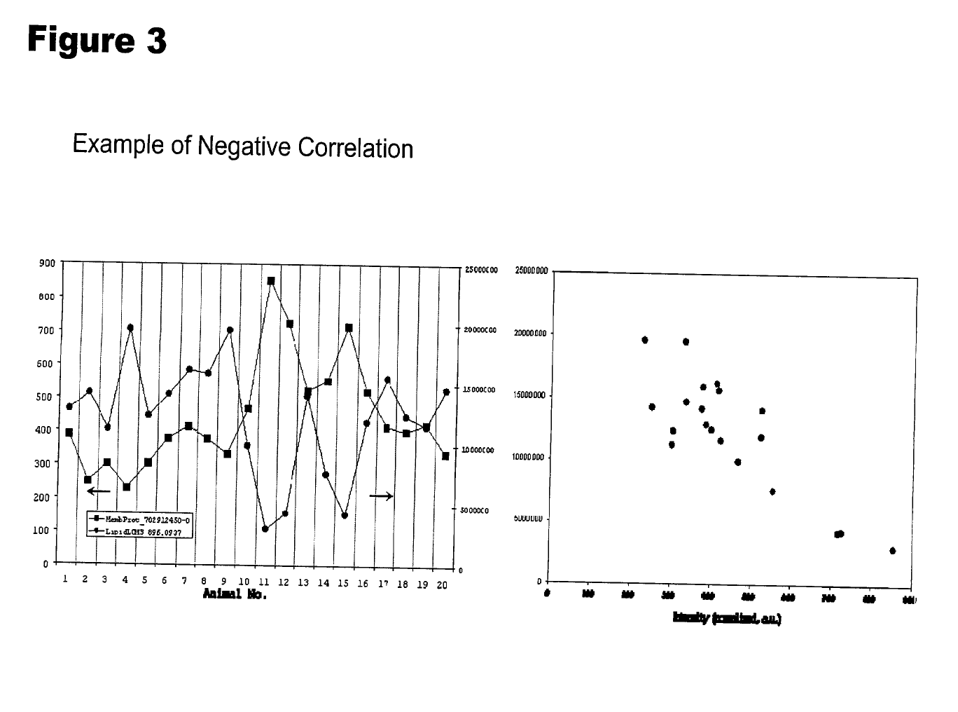

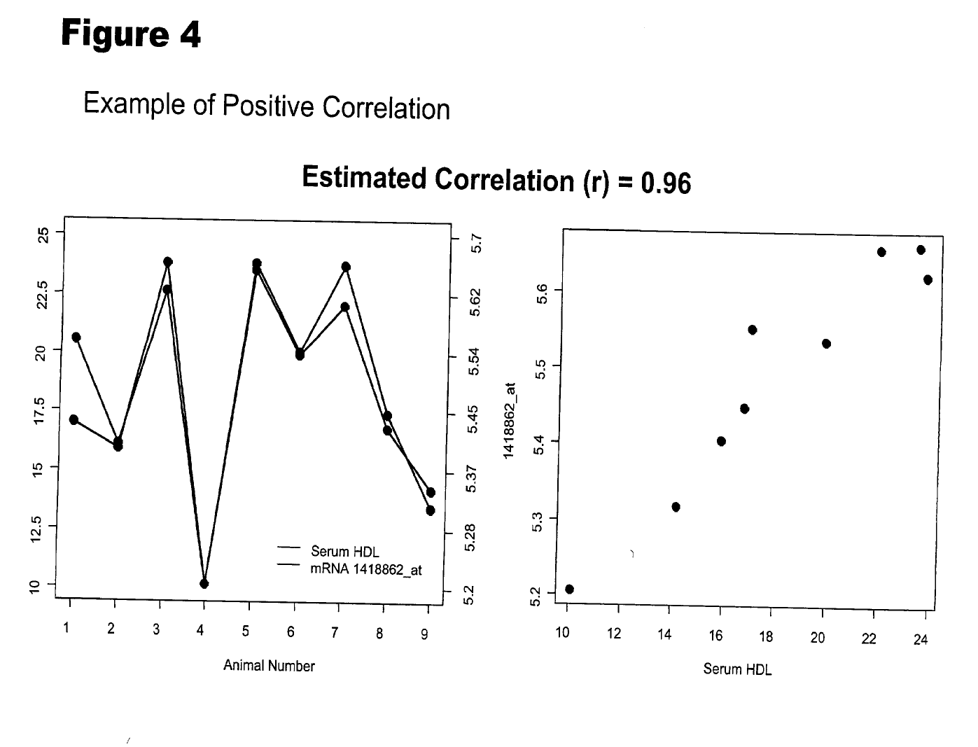

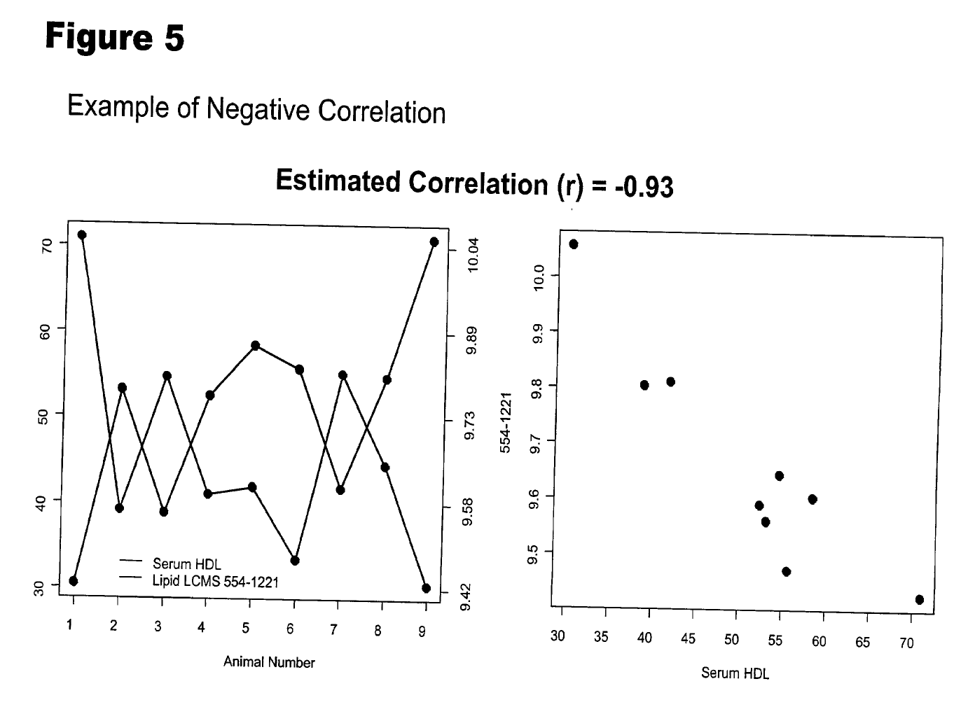

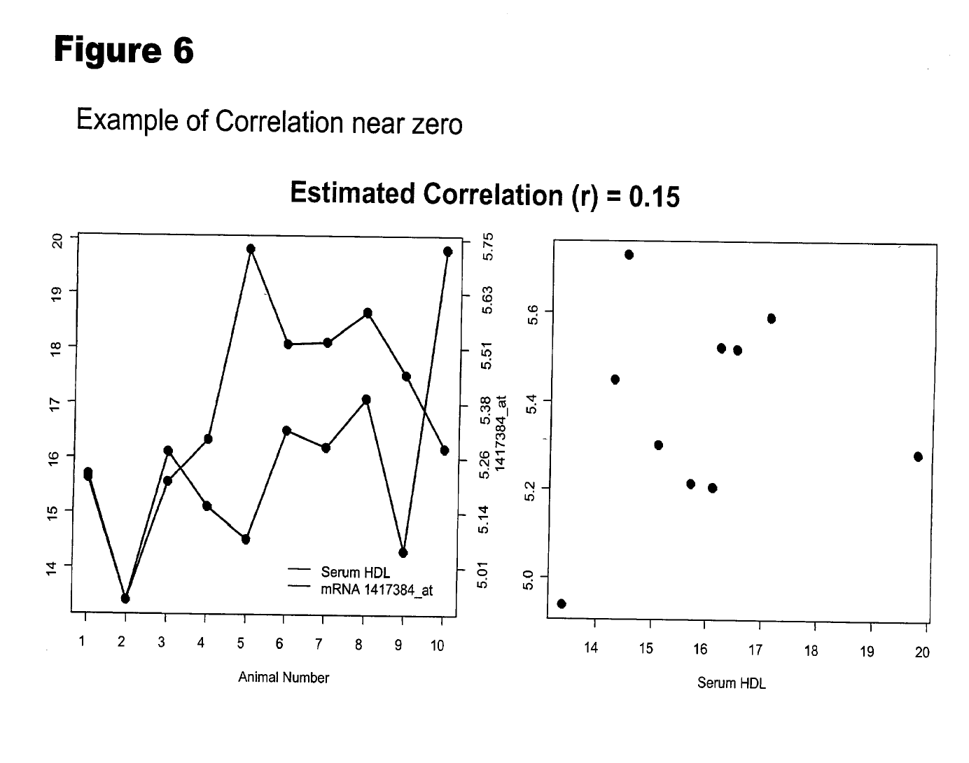

There are many methods to calculate a correlation between two measured features, including parametric analysis, non-parametric analysis, linear and non-linear correlation, Pearson's correlation analysis, Pearson's Product Moment Correlation analysis, Spearman rank correlation analysis, Kendall correlation analysis, and other statistical correlation methods known to those skilled in the art. The quantitative measure of correlation for a set of features is denoted by the Latin letter r. It is assumed that this measure is an estimate of the unobserved true correlation, ρ (Greek rho), in the entire population from which the samples for were obtained. Correlations are thus measures of the monotone behavior of pairs of features, and typically r takes on values such that −1≦r≦+1. When r is close to +1 the two analytes under study correlate well, in the sense that when the level of the first increases, so does the level of the second. See FIGS. 2 and 4. When r is close to −1 the two analytes also correlate well, but negatively (sometimes termed “anti-correlate” or an “anti-correlation”), in the sense that when the level of the first increases, the level of the second decreases. See FIGS. 3 and 5. When r is close to 0, the two analytes are said to be uncorrelated and their scatter plot will show no trend. See FIG. 6.

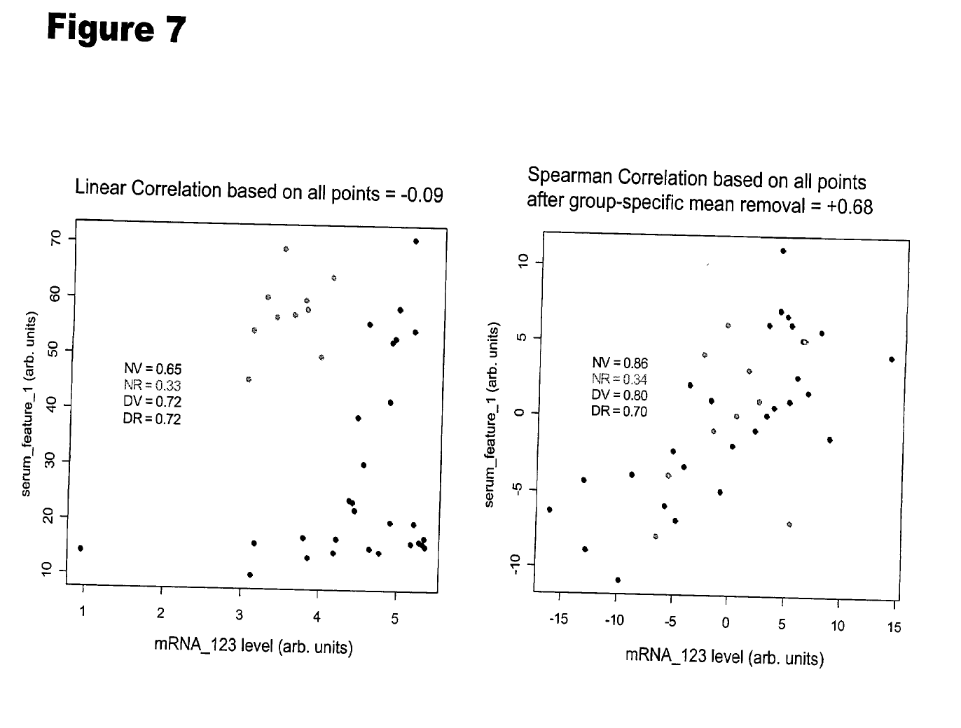

When analyzing datasets from multiple biological states of a system, calculating correlations between analytes can become confounded with the state-specific group mean of the data. Such a situation is shown in the left panel of FIG. 7, in which four distinct biological states are represented, each comprising ten animals. In FIG. 7, each data point represents the value of serum “feature l” (on the ordinate) and an mRNA “mRNA 123” (on the abscissa) for a given animal. When group membership of the animals is not properly taken into account (left panel of FIG. 7), a linear correlation yields a misleading value of r=−0.09 suggesting no correlation between this pair of features in the test animals.

One appropriate method of deconvolving group-specific effects from correlation calculations is the use of partial correlations. A partial correlation measures the strength of a relationship between two variables, while controlling the effect of one or more additional variables. The Pearson partial correlation for a pair of variables can be defined as the correlation of errors after regression on the controlling variables. In the present case, the variable to be controlled for is the mean values of the measurement values of serum feature 1 and mRNA 123 in each of the four groups. Upon subtracting these four group-specific means, the data are re-plotted as shown in FIG. 7, right panel, and a correlation (Spearman in the case of FIG. 7) can be calculated which is now not convolved with group-specific effects, and which therefore more accurately represents the association of the two measurements under study, and produces an r value of +0.68.

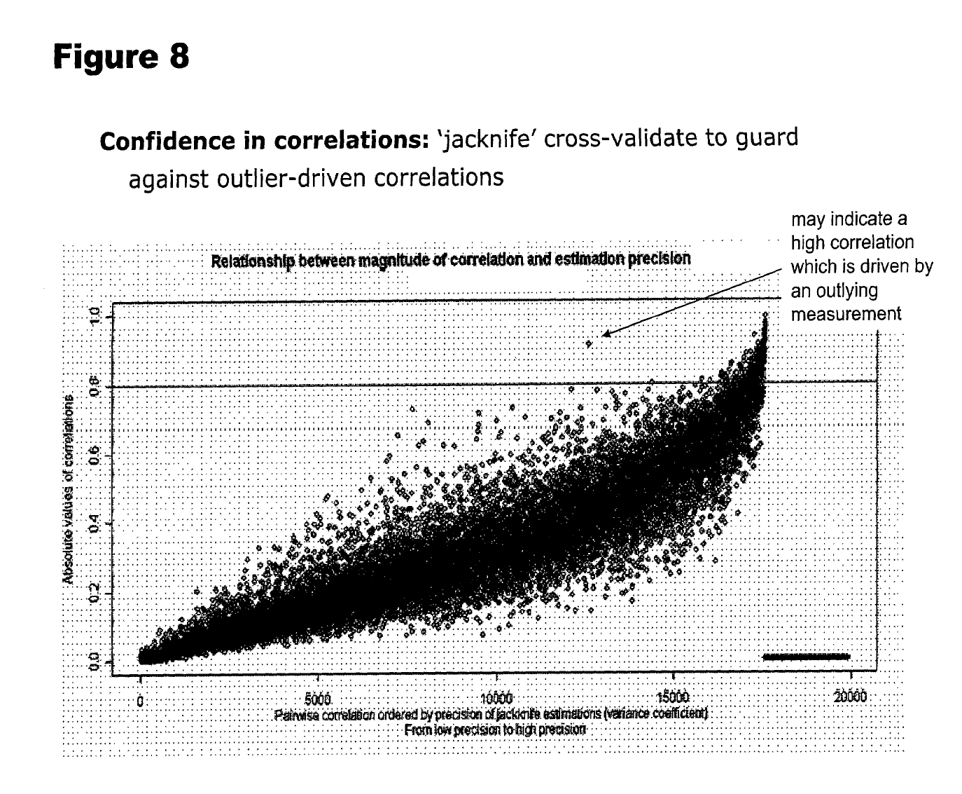

Furthermore, calculations of correlation values of two measurements can be undesirably and trivially influenced by one or a few samples which have anomalously measurement values different from than the rest of the measurements. To minimize such occurrences, each correlation calculation can be evaluated by a jack-knifing cross-validation routine, a representative result of which is shown in FIG. 8. Such a process is useful in identifying, e.g., levels of correlation which are spuriously high because of a measurement error or the like.

FIG. 1 also generally exemplifies another aspect of the present teachings, which permit development of data sets or profiles indicative or characteristic of a particular biological state of a particular biological compartment in an animal body. This is done by exploiting correlation analysis techniques disclosed herein to find correlations between data features present in an accessible body fluid which comprise a reliable surrogate for data features present in the cells of the organ or other body compartment, which features characterize the biological state under study. Thus, for example, as illustrated in FIG. 1, data features of biomolecules in plasma can be correlated with data features from biomolecules in liver. Correlation studies of course may be conducted using biomolecules from any two or multiple body compartments. This method can be used, for example, to develop blood tests suitable for determining development of a toxicity caused by administration of a xenobiotic before there are any overt symptoms of the toxicity. Such a method can enable prediction of the development of a particular biological state, e.g., efficacious response to a drug, before administration of the drug, or after administration of a sub-toxic micro-dose of a drug. This method also can be use to determine the biochemical relationship between any two or more body tissues in preselected biological states, for example, endothelial cells lining a vessel and blood.

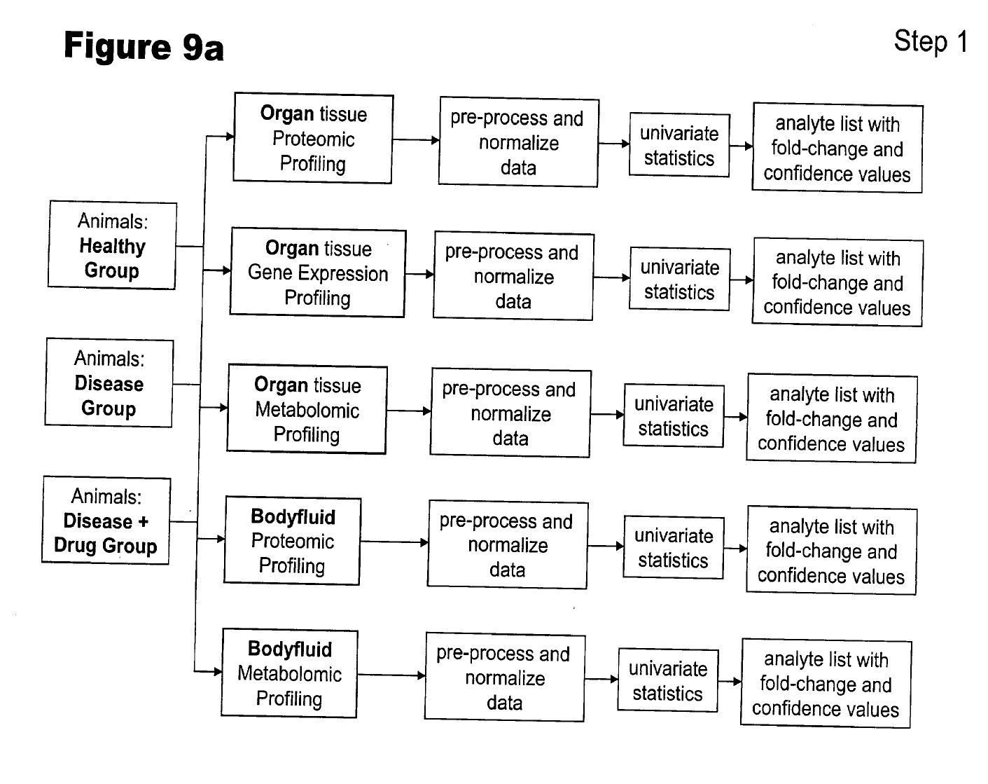

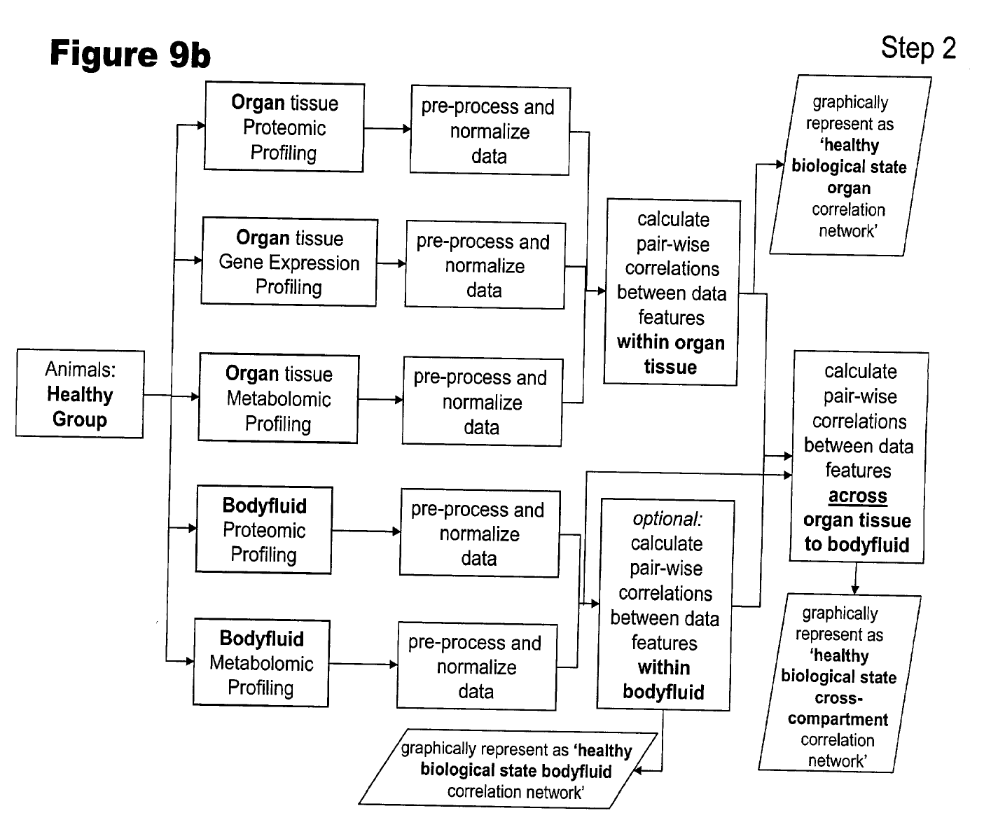

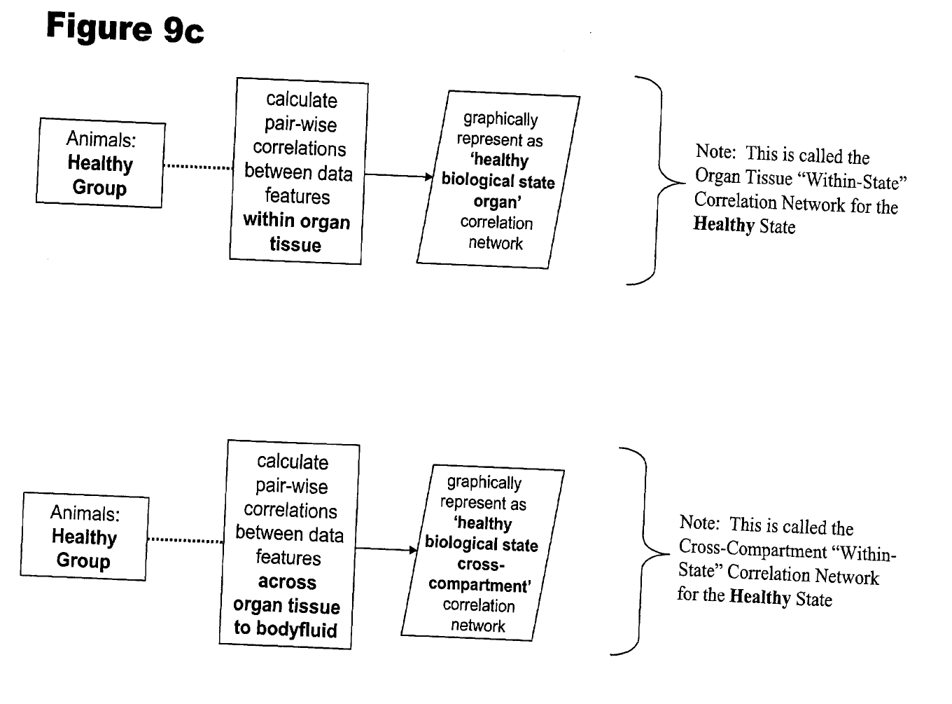

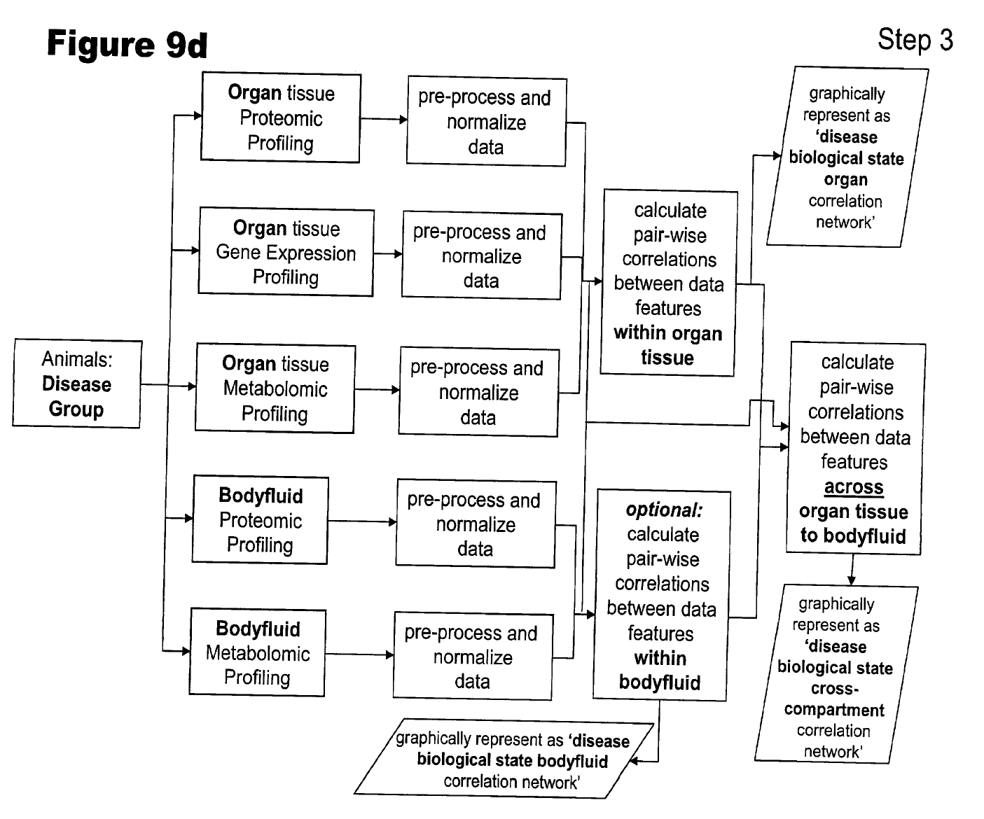

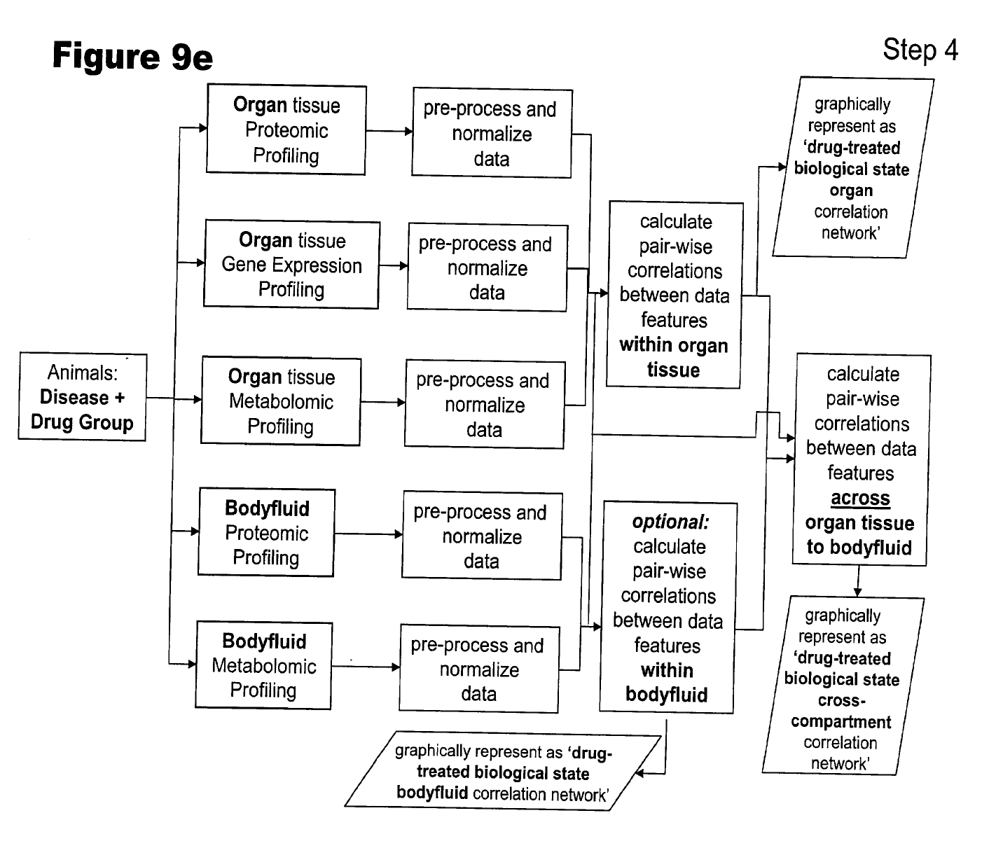

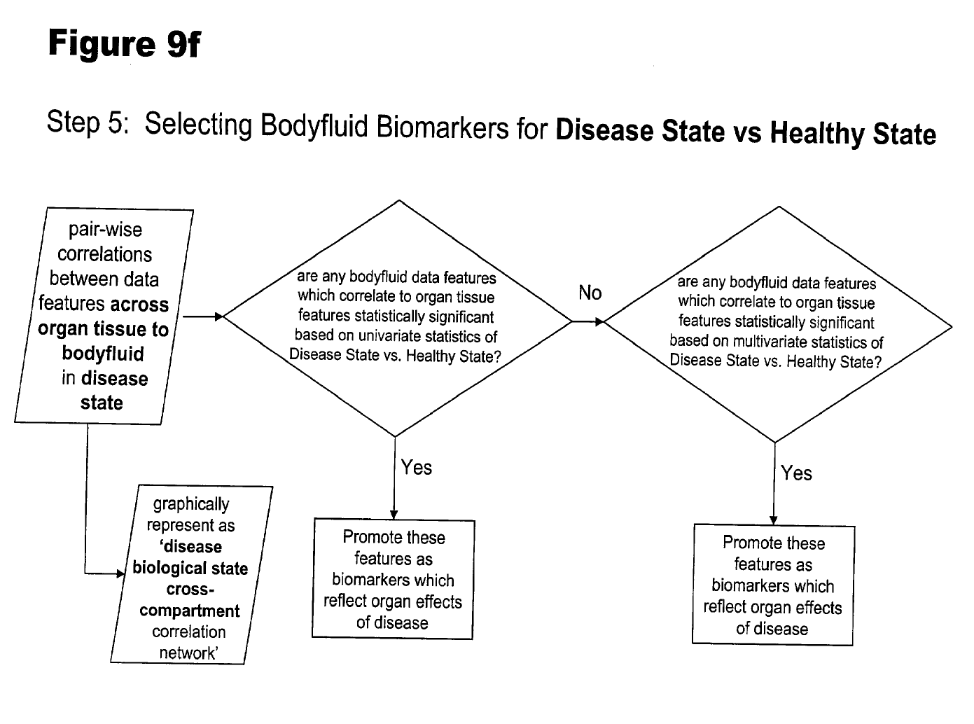

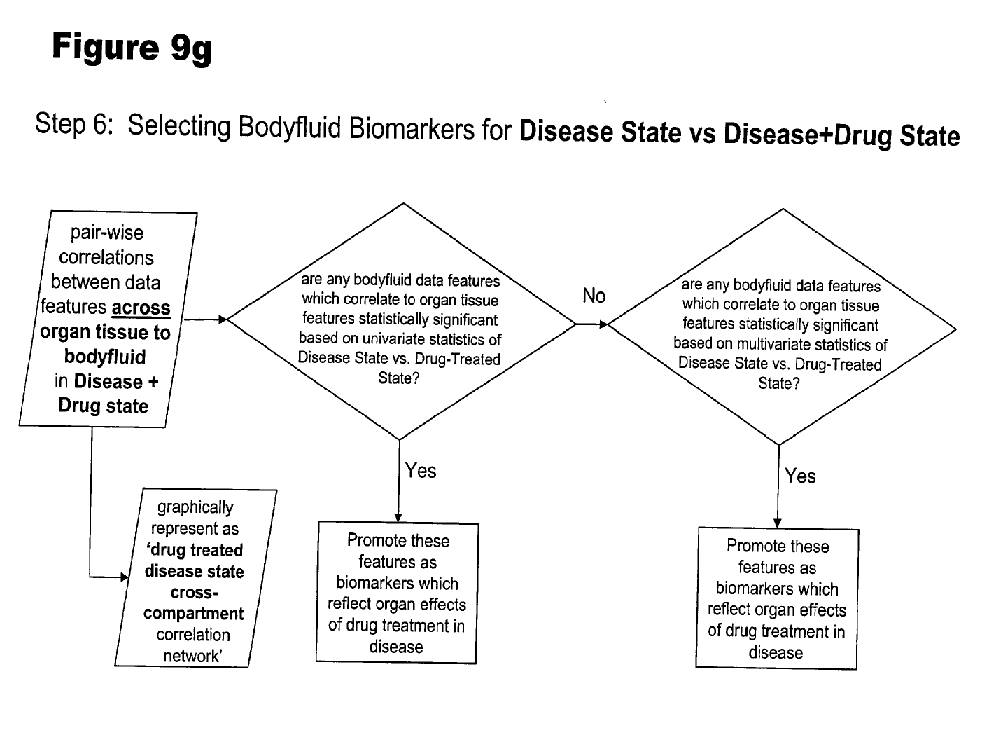

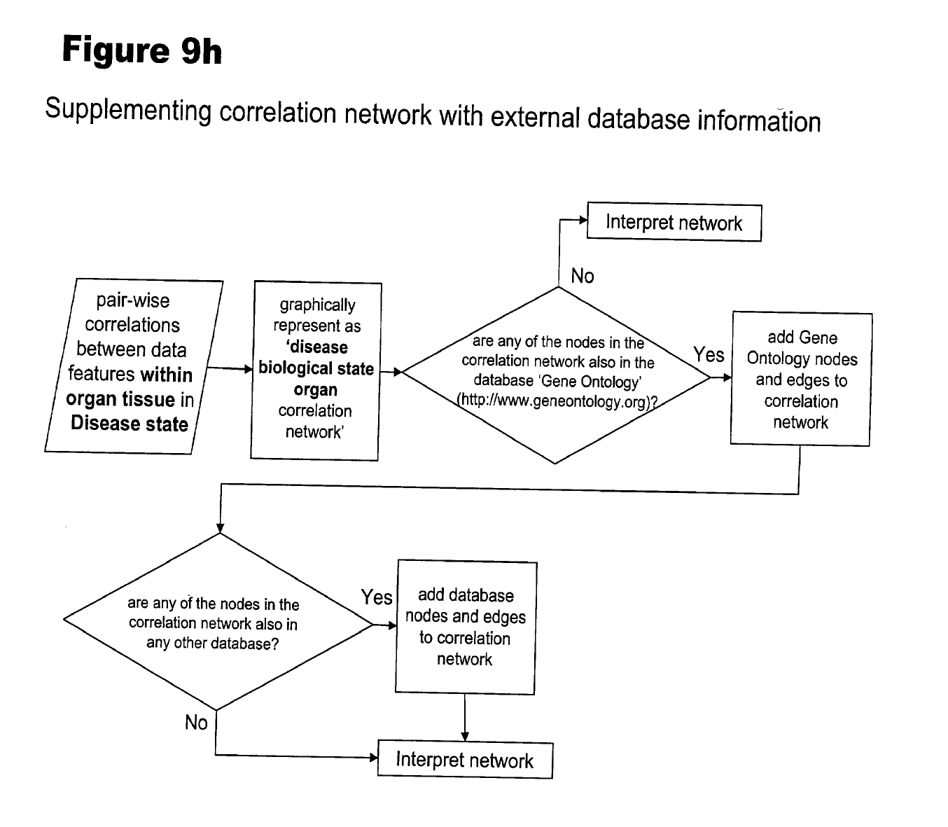

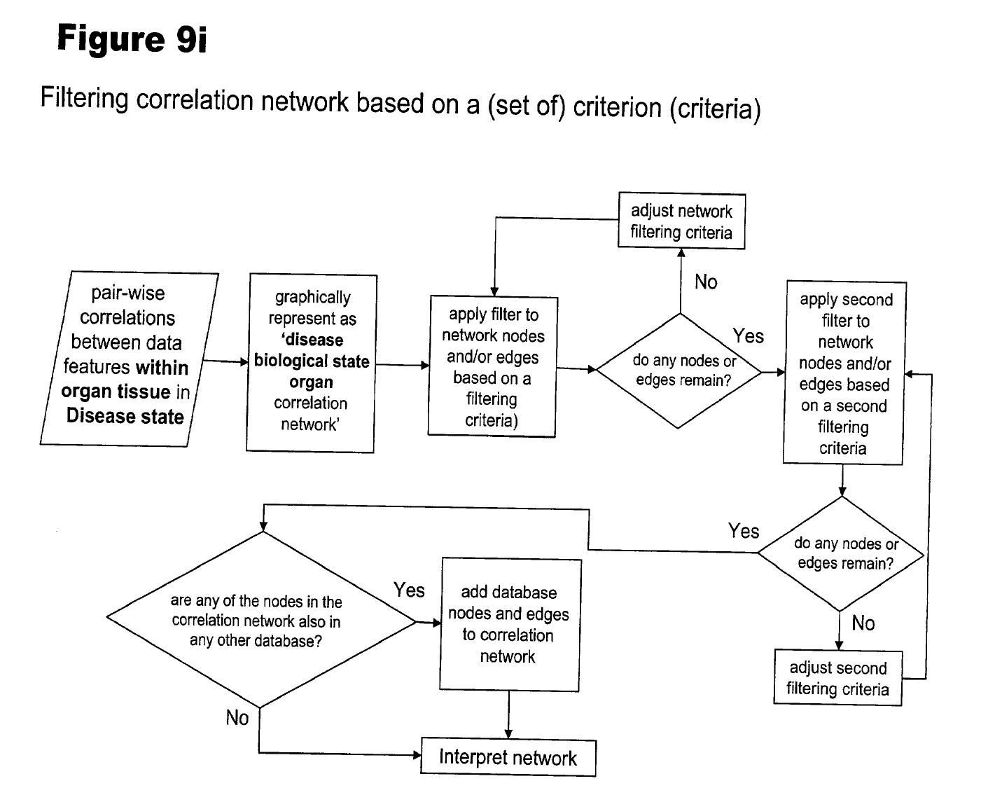

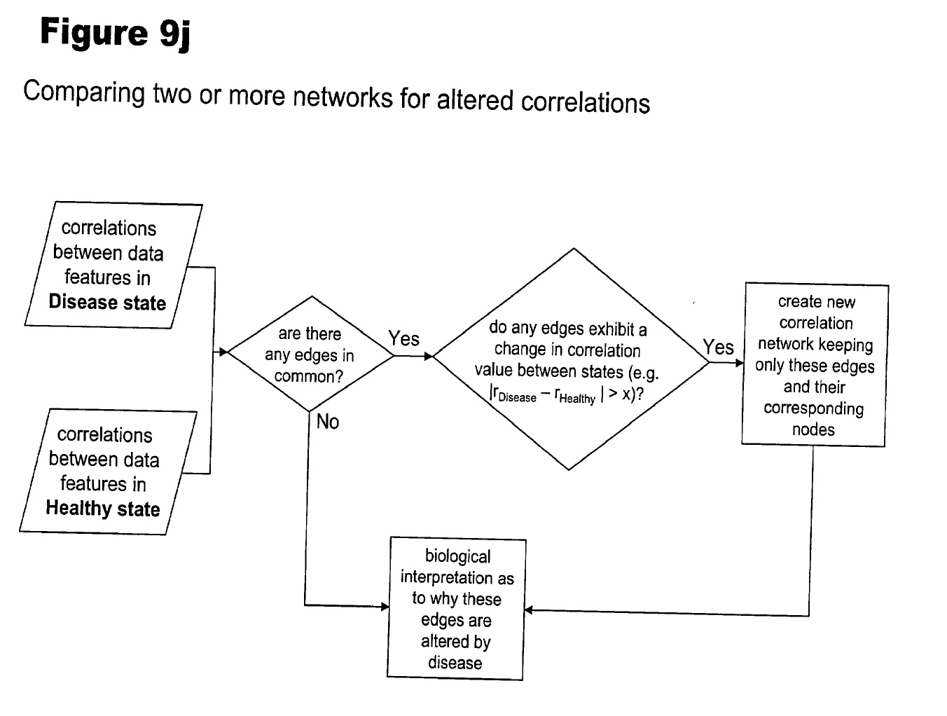

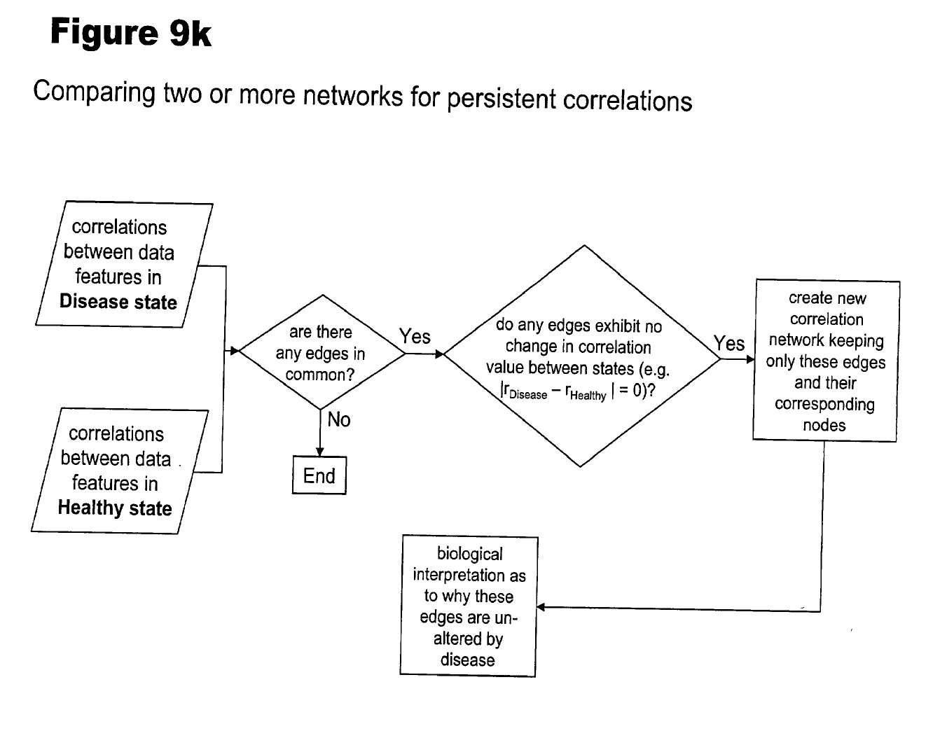

FIG. 9a through 9k are flow charts illustrating process steps that can be conducted in the practice of the present teachings, and are discussed below to further elucidate the present teachings. FIGS. 9a-9e depict flow diagrams illustrating generally various upstream operations. The operations can involve selecting animals, including human subjects, and, in appropriate cases, test and control subject groups. For each subject, one or more of various types of samples can be taken and analyzed for one or more types of biomolecules. These data then can be preprocessed and normalized so that valid comparisons among them can be done, and then the correlations, if any, can be detected. In some embodiments, the method begins with parallel analyses of mRNA, protein, and metabolite data sets derived from complex samples extracted from both diseased and healthy populations. The mean quantities, as well as the ranges and variances, for all measured compounds can be collectively analyzed using methods to identify molecules to link gene response, protein activity, and metabolite dynamics.

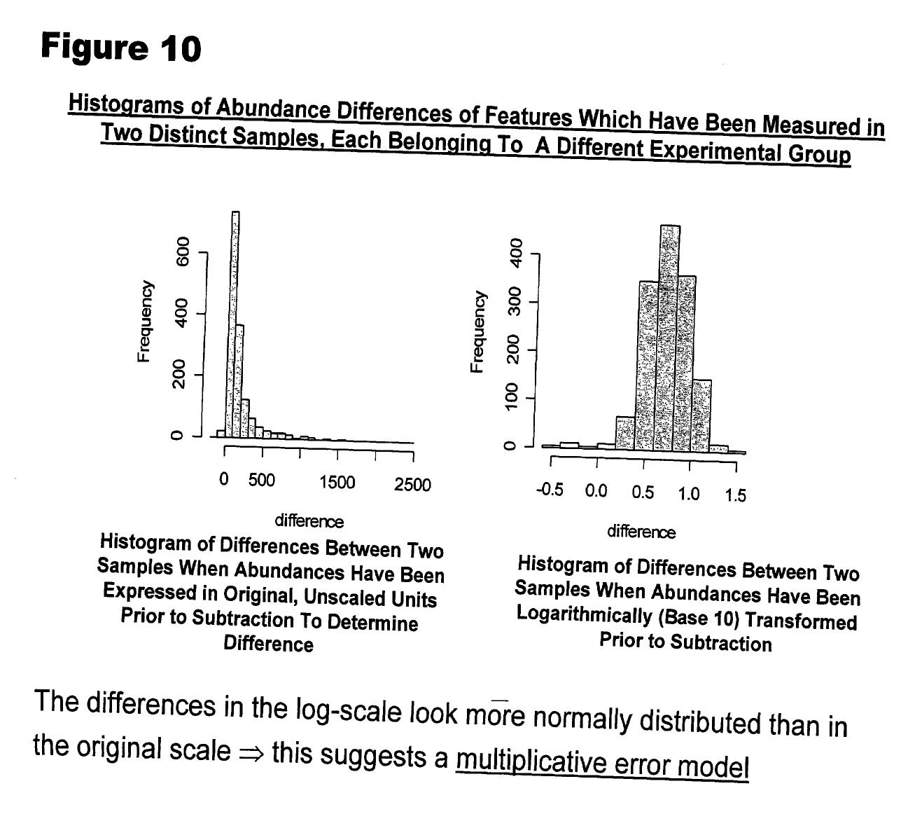

Referring to the pre-processing step in FIG. 9a, a method for evaluating whether dataset values need to be logarithmically transformed is now described. FIG. 10 represents histograms of differences in approximately one-thousand measured features across two samples; the left histogram considers feature difference values in the original scale, while the right panel shows the corresponding histogram after all data values have been logarithmically (base 10) transformed. As can be seen, the logarithmically transformed data appear to be more normally distributed, which can be verified by, for example, the Kolmogorov-Smirnov Test or other tests known to those skilled in the art. Furthermore, the fact that logarithmic transformation results in more normal distribution of measurement values indicates a multiplicative error model, relevant for the step of data normalization.

Referring back to the normalization step in FIG. 9a, a method for normalizing gene expression data, protein data, and metabolite level data is now described. Let Xᵢⱼ denote the raw measurement intensity, where i is the measurement index and ranges from i=1, . . . , I in a dataset with I measurements, and j is the sample index where j=1, . . . , J in a dataset with J samples. Based on the evaluation of the distribution of the data (FIG. 10), a multiplicative model is assumed:

Xᵢⱼ=mᵢ×rⱼ×eᵢⱼ,

and taking the logarithm results in

Yᵢⱼ=log(Xᵢⱼ)=μᵢ+ρⱼ+εᵢⱼ,

where it is assumed that εᵢⱼ is normally distributed with a mean of zero and variance σᵢ². The purpose of normalization is to estimate the multiplicative factors rⱼ=exp(ρⱼ) and to scale the data accordingly. The parameters ρᵢ, ρⱼ, σᵢ² are estimated by iterating the following equations until convergence:

$${\hat{μ}}_{i} = \frac{1}{J} {\sum }_{j = 1}^{J} ( {Y}_{ij} - {\hat{ρ}}_{j} )$$

$${\hat{ρ}}_{j} = \frac{{\sum }_{i = 1}^{I} {( {\hat{σ}}_{i}^{2} )}^{- 1} ( {Y}_{ij} - {\hat{μ}}_{i} )}{{\sum }_{i = 1}^{I} {( {\hat{σ}}_{i}^{2} )}^{- 1}}$$

$${\hat{σ}}_{i}^{2} = \frac{1}{J} {\sum }_{j = 1}^{J} {( {Y}_{ij} - {\hat{μ}}_{i} - {\hat{ρ}}_{j} )}^{2} .$$

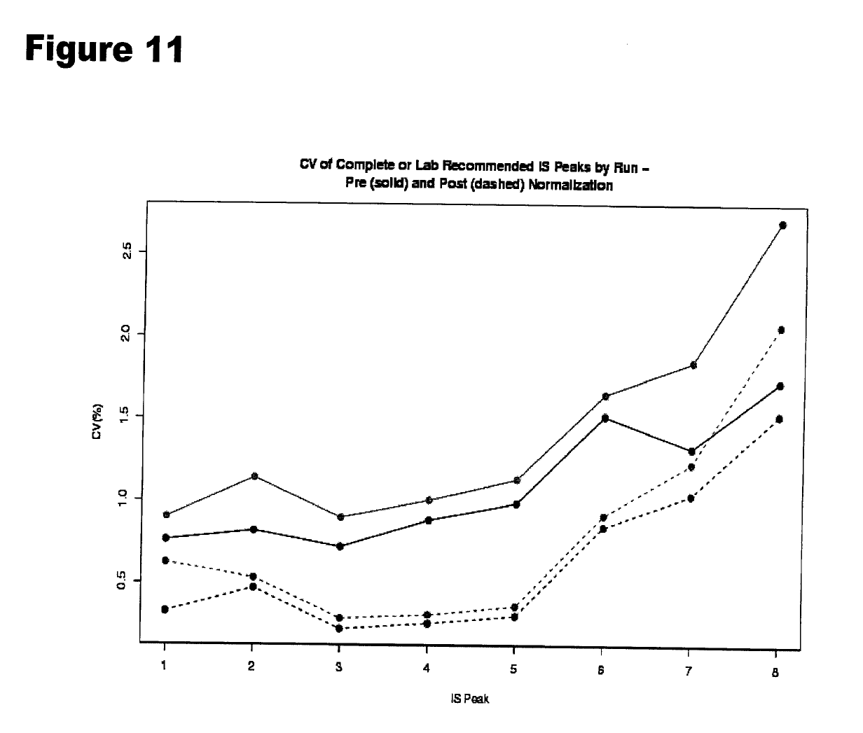

Many different procedures, such as a maximum likelihood iterative estimation, can be used to estimate the parameters. FIG. 11 illustrates coefficients of variance as determined from all samples for 8 measurements from an LC-MS analytical platform before normalization (solid lines) and after normalization (dashed lines), showing the desired effect of normalization in generally decreasing coefficients of variance.

The normalized data can be compared to a null model, and a p-value can be calculated that measures the probability that the deviation of the data from the null model can be attributed to the random error. The parameter used for comparison is the fold ratio between the two chosen varieties. To evaluate the method, a t-test can be performed to compare the two chosen varieties. (D. J. Sheskin, Handbook of Parametric and Nonparametric Procedures, Chapman & Hall/CRC, Boca Raton, Fla. (2000)). The corresponding p-values were calculated for each gene. When assessing the statistical significance of fold change for each gene, the total N_{g} p-values calculated should be considered, as several p-values with p<1/N_{G} are expected. To account for this, the overall likelihood, P(p), of observing a p-value ≦p for any of the N_{g} genes can be used. Assuming independence of all genes, the overall likelihood is estimated with:

P(p)≈1−(1−p)^{N}^{_{G}}.

Assuming independence of genes is obviously an oversimplification. The correct way to calculate p-values and P(p) values is by using the bootstrap method with the parameters of the null model being used to general random data sets.

Returning again to FIG. 9a, the application of univariate statistics results in analyte lists disclosing fold changes and optionally confidence levels. It is not necessary in the analysis at this stage to know the identity of the biomolecules in the list, but only that one is looking at the same biomolecule (or a species homolog) in the samples and groups being compared. From these data, positive and negative correlations can be assessed across, for example, healthy, diseased and drugged diseased groups, to elucidate groups of values or a collection of data that characterize, i.e., identify uniquely or with high probability, a given biological state.

FIG. 9b illustrates an embodiment of the present teachings wherein a correlation data set between a body fluid and an organ is developed for a healthy animal. FIG. 9c illustrates organ (tissue) and cross compartment analyses protocols for healthy animals. FIG. 9d illustrates an embodiment of the present teachings wherein a correlation data set between a body fluid and an organ is developed for a diseased animal. FIG. 9e illustrates an embodiment of the present teachings wherein various correlation data sets are developed for untreated diseased animals and drugged diseased animals, within a body fluid, within an organ, and between a body fluid and an organ. Such analyses can be useful in drug development as disclosed herein. FIG. 9f is a block diagram depicting the general approach to developing a profile or biomarker for distinguishing biological states, e.g. a diseased state vs. a healthy state, so as to permit determination of the state at the organ or tissue from correlated surrogate markers found in a body fluid. FIG. 9g illustrates an approach similar to that shown in 9f, except that untreated and drug treated groups are analyzed to develop biomarkers.

FIGS. 9h-9k illustrate additional operations that can be done to probe biological states in various ways. FIG. 9h illustrates supplementing correlation network analyses with external database information; 9i illustrates filtering correlation networks based on one or a set of criteria; FIG. 9j illustrates comparing two or more networks for altered correlations; and FIG. 9k illustrates comparing two or more networks for persistent correlations.

### Exemplification

### Systems Biology Analysis of Mouse Model of Disease and Drug Treatment

As a test case for the application of systems biology analysis to a mammalian system, an animal experiment was performed to profile biological systems in (i) a healthy state administered a non-therapeutic placebo vehicle (denoted as the “normal vehicle” state, and abbreviated herein as the “NV” state), (ii) a disease state administered a non-therapeutic placebo vehicle (denoted as the “disease vehicle” state, and abbreviated herein as the “DV” state), (iii) a disease state treated by a therapeutic drug (denoted as the “disease treated” state, and abbreviated herein as the “DR” state), and (iv) a healthy state treated by a therapeutic pharmaceutical drug (denoted as the “normal treated” state, and abbreviated herein as the “NR” state).

The animals in this experiment were C57BL/6 mice. Ten different animals were used per each of the four biological states enumerated above. To induce disease, the mice in the DR and DV groups were fed a diet enriched in fat, while the mice in the NV and DV groups were fed a relatively lower fat diet.

After eight weeks of growth on the diets indicated above, the animals in each group were administered a two-week course of either the therapeutic drug (for the NR and DR states) or the non-therapeutic placebo vehicle (for the NV and DV states). All animals were then sacrificed and terminal blood and adipose tissue was collected.

Tissue samples from all animals were analyzed to assess gene transcriptional activity. Endogenous metabolite levels were determined from both blood serum and adipose tissue.

### Tissue Gene Transcriptional Analysis

Transcriptional analysis of genes and expressed sequence tags provides valuable information about biological processes. Affymetrix GeneChip® technology (Affymetrix, Santa Clara, Calif.) measures such changes. In brief, this technology uses messenger ribonucleic acid (mRNA) from an experimental condition to obtain complementary deoxyribonucleic acid (cDNA), and ultimately, complementary ribonucleic acid (cRNA) for hybridization to Genechip® arrays. Genechips® contain nucleic acid probes for thousands of sequences that are bound to a solid surface. Affymetrix Genechip® technology was used to assay transcriptional changes in the tissues in this study. Extracted mRNA samples were hybridized to the GeneChip® Mouse Genome 430A Array. Relative mRNA intensity levels for >22,000 probe sets were obtained using Affymetrix® Microarray Suite version 5.0 (MAS 5.0, Affymetrix, Santa Clara, Calif.). The processed data were log-transformed (base 10) prior to subsequent data analysis.

Profiling of Lipids Extracted from Adipose Tissue and Blood Serum

Serum samples were aliquoted in duplicate into 10 microliter aliquots for liquid chromatography-mass spectrometry (LC-MS) lipid analysis. Prior to aliquoting, digital photographs were taken of thawed serum samples. Organic solvent containing three internal standards (17:0 lysophosphatidylcholine, symmetric 12:0 phosphatidylcholine, and symmetric 17:0 triglyceride) were added to the serum and the resulting supernatant was used for LC-MS analysis.

Tissue samples were manually cut into 2 equivalent pieces whose masses ranged from 12 mg to 28 mg. The tissue pieces were added to tubes containing H₂O and ceramic beads. The samples were then treated with focused acoustic energy and snap frozen on dry ice. The frozen samples were lyophilized and extracted with organic solvent containing three internal standards (17:0 lysophosphatidylcholine, symmetric 12:0 phosphatidylcholine, and symmetric 17:0 triglyceride). The resulting supernatant was used for LC-MS analysis.

LC-MS analysis was performed on a Waters/Micromass quadrupole time-of-flight instrument (Q-ToF Micro, Waters/Micromass, Milford, Mass.) equipped with Lock Spray over a range of 200 to 1300 m/z. A Waters Alliance HPLC system (Waters/Micromass, Milford, Mass.) was used to separate and deliver analytes to the mass spectrometer.

The raw data was peak picked and integrated by IMPRESS software (proprietary software, BG Medicine, Inc., Waltham, Mass.). Alignment of analyte peaks for quality control and statistical analysis was performed by EQUEST software (proprietary software, BG Medicine, Inc., Waltham, Mass.). Quality control was assessed by calculating the percent relative standard deviation (% RSD) of all three internal standards across a replicate and plotting the internal standard areas as a function of time to identify any trends. The % RSD for the 17:0 lysophosphatidylcholine, the 12:0 phosphatidylcholine, and the 17:0 triglyceride internal standards were under 10%, under 10% and under 35%, respectively. No significant trending of internal standards was observed over the course of any replicates. Because of peak misalignments or below limit of detection measurement, missing values frequently appeared. Those peaks with 50% or more missing values in at least one of the several disease-by-drug groups were excluded from the analysis.

The processed data were log-transformed (base 10) and a constant “1” was added to all data (prior to log-transformation due to 0's in the data) before data analysis.

For peak identification, LC-MS/MS analysis was performed on a Waters/Micromass quadrupole time-of-flight instrument (Q-ToF Micro, Waters/Micromass, Milford, Mass.) equipped with Lock Spray over a fragment ion range of 50 to 1300 m/z. A Waters Alliance HT LC system (Waters/Micromass, Milford, Mass.) was used to separate and deliver analytes to the mass spectrometer. Prioritized analyte identifications were manually deisotoped prior to being added to an inclusion list on the instrument. Spectra obtained were identified using a combination of reference spectra, elemental composition calculations, signature ions, retention time, and manual interpretation.

### Univariate Statistical Analysis

For all standard statistical tests, p-values were generated for each measurement and each treatment comparison. In general, an alpha-level of 0.05 without any adjustment cannot be used as an indication of statistical significance when multiple hypothesis tests are performed. P-values were adjusted for False Discovery Rate (FDR) based on the approach of Benjamini (Y. Benjamini and Y. Hochberg, “Controlling the false discovery rate: A practical and powerful approach to multiple testing,” Journal of the Royal Statistical Society (Series B), 57(1):289-300 (1995)).

The median fold change (MFC) of a measurement, which represents the median amount of change in one group compared to the other, was calculated for measurements, along with FDR-adjusted p-values. The median fold change was calculated as follows: (i) if the median value of the experimental group (drug or diseased) is greater than the median value of the control group (vehicle or normal), then median fold change is the median value of experimental group divided by the median value of the control group, and the direction of the median fold change was denoted as ‘Increased’, or ‘I’; and (ii) if the median value of the experimental group (drug or diseased) is less than the median value of the control group (vehicle or normal), then median fold change is the median value of control group divided by the median value of the experimental group.

The primary univariate analysis was based on analysis of variance (ANOVA). The ANOVA model included main effects (drug and disease) and two factor interaction (drug-by-disease).

### Correlation Analysis

Correlation networks in this study are graph representations of sets of pair-wise mathematical correlations between intensity values of measured analytes. The types of correlations performed in the present study included Pearson and Spearman rank-order correlations. The formula for calculating Pearson correlation is:

$$r = \frac{{\sum }_{i = 1}^{n} {x}_{i} {y}_{i} - \frac{1}{n} {\sum }_{i = 1}^{n} {x}_{i} {\sum }_{i = 1}^{n} {y}_{i}}{\sqrt{{\sum }_{i = 1}^{n} {x}_{i}^{2} - \frac{1}{n} {( {\sum }_{i = 1}^{n} {x}_{i} )}^{2}} \sqrt{{\sum }_{i = 1}^{n} {y}_{i}^{2} - \frac{1}{n} {( {\sum }_{i = 1}^{n} {y}_{i} )}^{2}}}$$

where r is the correlation coefficient, xᵢ is the i^{th} measurement of feature x, yᵢ is the i^{th} measurement of feature y, and n is the total number of samples in which x and y were measured. Note that the n samples may be n different animals, n different times, n different drug dosages, etc. In the present case, n samples are n different animals. The hypothesis that the correlation is zero (r=0) is tested using the formula:

t=[r*SQRT(n−2)]/[SQRT(1−r²)]

where r is the correlation coefficient, n is sample size, and the t value is looked up in a table of the distribution of t, for (n−2) degrees of freedom. If the computed t value is as high or higher than the table t value, then the conclusion is the correlation is significant (that is, significantly different from 0).
Spearman rank-order correlation is a nonparametric measure of association based on the rank of the data values. The formula is:

$$θ = \frac{\sum ( {R}_{i} - \overline{R} ) ( {S}_{i} - \overline{S} )}{\sqrt{\sum {( {R}_{i} - \overline{R} )}^{2} \sum {( {S}_{i} - \overline{S} )}^{2}}}$$

where Rᵢ is the rank of the ith x value, Sᵢ is the rank of the ith y value, R_bar is the mean of the Rᵢ values, and S_bar is the mean of the Sᵢ values.

In correlation network graphs, measured analytes are represented by ‘nodes’, and correlations between pairs of analytes are represented by links, or ‘edges’, which connect the corresponding nodes. Correlations can be derived for pairs of analytes measured either within or across tissues or compartments. In addition, measurements from diverse platforms such as gene expression and LC-MS can be integrated by examining correlations between and among such analyte measurements. In the correlation network graphical representation, each analyte is represented by a node and is assigned a co-ordinate in a two-dimensional plane. Further, the polygonal shape of a node represents the bioanalytical platform on which it was measured.

The quantitative measure of correlation for a set of data is denoted by the Latin letter r. It is assumed that this measure is an estimate of the unobserved true correlation, ρ (Greek rho), in the entire population from which the samples for the present study were obtained. When r is close to +1, the two analytes under study correlate well in the sense that when the level of the first increases, so does the level of the second. When r is close to −1, the two analytes anti-correlate well in the sense that when the level of the first increases, the level of the second decreases. When r is close to 0, the two analytes are said to be uncorrelated and their scatter plot will show no trend.

Based on the estimated correlations (r), statistical tests are performed for testing ρ=0; only correlations significantly different from 0 are represented as edges in a graphical correlation network. A false discovery rate (FDR) correction is implemented in the estimation of r.

One sub-type of correlation network analysis which was pursued is termed “within-state” correlations. These refer to correlation calculations performed on data derived from the group of animals representing a single biological state. For within-state correlations, Pearson correlations were calculated between pairs of normalized, un-transformed (i.e. original units) peak intensities derived from measurements.

Another sub-type of correlation network which was pursued is the network type termed the “across-state”. In this case, a correlation value between any two analytes is calculated using the data for that pair of analytes from all four animal groups, representing the four biological states of the current study. Explicitly, the four states of the study are NV, DV, DR, and NR.

The general approach to constructing a correlation network is to determine firstly all pairs of correlations among the set of measured analytes, independent of tissue or platform type. Subsequently, select subsets of the correlation network may be further displayed and explored. Interesting subsets may be chosen based on nodes which exhibit significant univariate median fold changes, nodes which are known to be associated with the disease or drug state under study.

### Traversing Biological Data Sources

From a set of identified analytes, relationships to known biological observations through the use of database traversals can be determined. These traversals create new edge representations on correlation network graphs which reflect a new type of connectivity. For example, if a gene transcript and its protein product are both found on a correlation network, the edge connecting them is of the type transcription-translation. The first traversals undertaken are typically done through the biological process and cellular component hierarchies of Gene Ontology (www.geneontology.org) as a way of putting the correlation networks into biological context. Using this approach it is possible to demarcate subgraphs of the network to address questions such as: What are the secreted proteins in this network? Or what transcripts code for transcription factors? Other traversals included metabolism databases such as KEGG, (Kanehisa M, Goto S, Kawashima S, Nakaya A., The KEGG databases at GenomeNet, Nucleic Acids Res, 30:42-6, (2002)) to place identified metabolites in biological context, and PubMed to connect any identified analyte pair through literature co-occurrence.

For this study, traversals through the biological process and cellular component hierarchies of Gene Ontology and the KEGG compound database were performed to link these networks to correlation networks. These traversals helped to classify correlation networks into their primary biological processes, cellular locations and metabolic pathways.

### Identifying and Elucidating Key Sub-Networks

If correlation networks have a high node and edge count, generally above a few hundred of each, then they are examined for sub-networks or network motifs. This network motif analysis can focus on a few principles: (1) important a priori known analytes in the disease state and their neighboring nodes are areas of focus; (2) correlations which exhibit change upon disease or treatment—“state-change” networks—are of interest, as they may be revealing disease or drug processes; and (3) highly inter-connected nodes (e.g., those characterized by high node degree or high clustering coefficient) and their neighbors, both potential properties of “hubs” in scale-free networks, are of interest as they may be expose novel insights into disease or treatment mechanisms (A. Vázquez, R. Dobrin, D. Sergi, J.-P. Eckmann, Z. N. Oltvai, and A.-L. Barabási, “The topological relationship between the large-scale attributes and local interaction patterns of complex networks,” Proc. Natl. Acad. Sci. USA, 101(52):17940-17945, (2004)). All three approaches were used in the present study.

### Application of Correlation Analyses

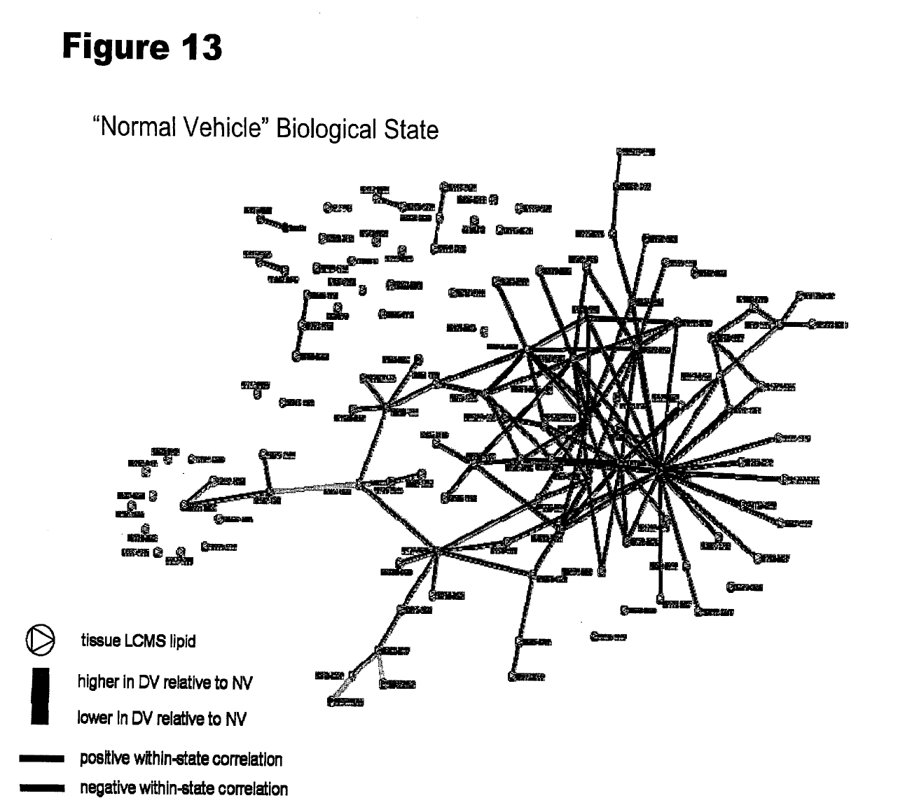

A set of correlations as graphically represented by a correlation network or a subset of such a correlation network constitutes a profile of a biological state. As a reminder, the four biological states in the current study are NV, DV, DR, and NR. For example, FIG. 12 represents a correlation network in liver tissue, with all measured analytes as nodes, in the DV biological state, with the condition that a correlation edge is shown if the correlation between a pair of measurements (as represented by nodes) has a Pearson's correlation value of |r|≧0.8; this results in a correlation network with approximately 3,400 nodes and 17,000 edges. Analogous networks for the remaining biological states in this experiment have also been produced. Further, FIGS. 13, 14, and 15 represent subsets of a larger correlation network of the type exemplified in FIG. 12 in three of the biological states in the current study: NV, DV, DR. The construction of these sub-networks is described below.

### “State-Change” Correlation Networks

To characterize and create a profile for each of the four biological states in this experiment, correlation networks were calculated and generated which exhibit statistically significant change between these four states. These are termed “state-change” correlation networks. In the present study, individual correlation networks were calculated for each of the four groups of animals in the study. State-change networks are particularly helpful in determining and evaluating the correlation changed induced by a disease or drug intervention.

The state-specific correlation networks are termed “within-state” networks. For state-change networks, the within-state correlation value of a given link, also termed “edge,” is compared across the DV, NV and DR within-state networks; this edge is kept in the final state-change network only if it exhibits a statistically significant change in correlation value induced by disease (determined by comparing the value of that correlation in the NV and DV states) or induced by treatment (determined by comparing the value of that correlation in the DV and DR states). These conditions were chosen because two of the primary foci of the present study were disease effect and treatment effect. For within-state networks, Pearson correlations were calculated between pairs of normalized and un-transformed peak intensities derived from analytes profiled by bioanalytical platforms. Finally, for the state-change network, a change in correlation of an edge between pairs of states was statistically tested using the following statistical null hypothesis (H₀):

Ho:ρₛₜₐₜₑ₁(i,j)=ρₛₜₐₜₑ₂(i,j)

where ρₛₜₐₜₑ₁ is the population correlation within a state (e.g. as within all normal vehicle animals) and ρₛₜₐₜₑ₂ is the population correlation within a second state (e.g. as within all disease vehicle animals), and i, j denote the ith and jth analytes, measured in both states. This statistical test of the null hypothesis generates both an estimated value of the population correlation change as well as an associated probability p-value which is subsequently adjusted for multiple hypothesis testing. For example, one statistical analysis used to test the null hypothesis is the well-known Fisher z-transformation of the correlation coefficients, z=(0.5)*[log((1±c1)/(1−c1))−log((1+c2)/(1−c2))]/sqrt[(1/(N1−3))+(1/(N2−3)), where c1 is the correlation within state 1, c2 is the correlation within state 2, N1 is the number of samples used to calculate the correlation within state 1 (N1=10 in this example), and N2 is the number of samples used to calculate the correlation within state 2 (N2=10 in this example).

As stated above, each state-change network was constrained to those correlations which exhibited a statistically significant change in the comparison of NV and DV states, or DV and DR states; explicitly the null hypotheses H0:ρNV(i,j)=ρDV(i,j) and H0:ρDV(i,j)=ρDR(ij) were statistically tested based on the set significance level. A further constraint imposed in these state-change networks was that the difference between (r_{NV(i,j)}−r_{DV(i,j)}) and (r_{DV(i,j)}−r_{DR(i,j)}) must have been of opposite sign; this criteria was set with the intention of selecting only those correlations which were altered in the DV state and tended to change in the direction of restoration toward the value in the NV state upon drug treatment (the DR state).

The process of correlation network calculation can be automated. Automation can eliminate the need for any extensive manual network calculations as all calculations are performed on the appropriate data sets in the appropriate database environment.

FIGS. 13, 14, and 15 are state-change networks in which only tissue LCMS lipid measurements were considered as input to the correlation network calculations. In these figures, only correlation edges with a Pearson correlation coefficient of |r|≧0.9 are included.

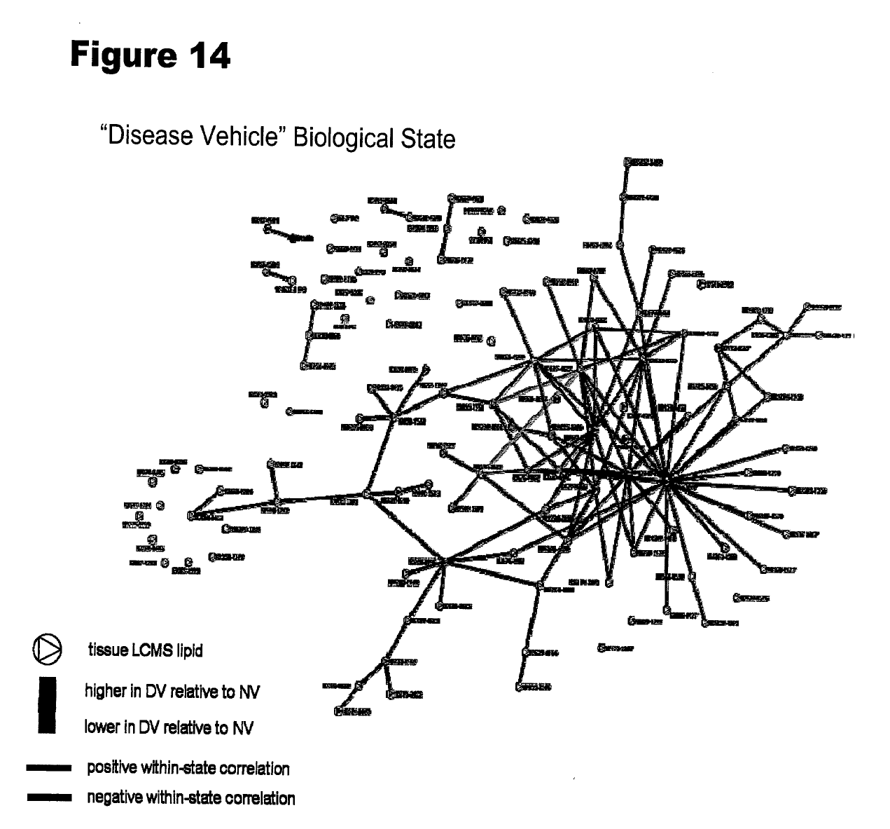

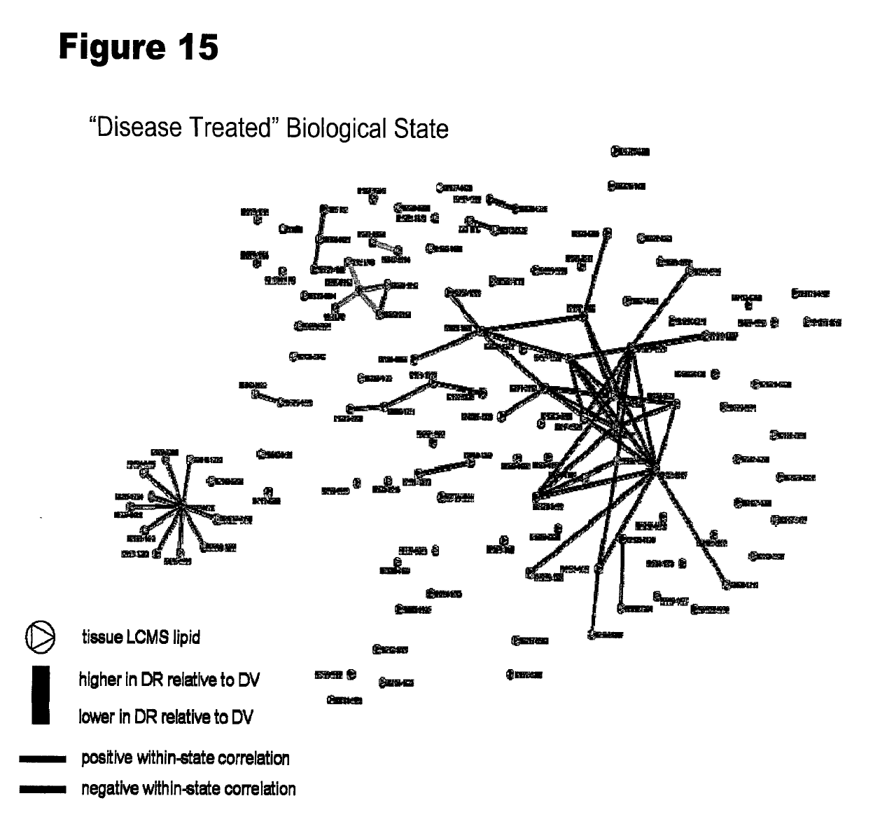

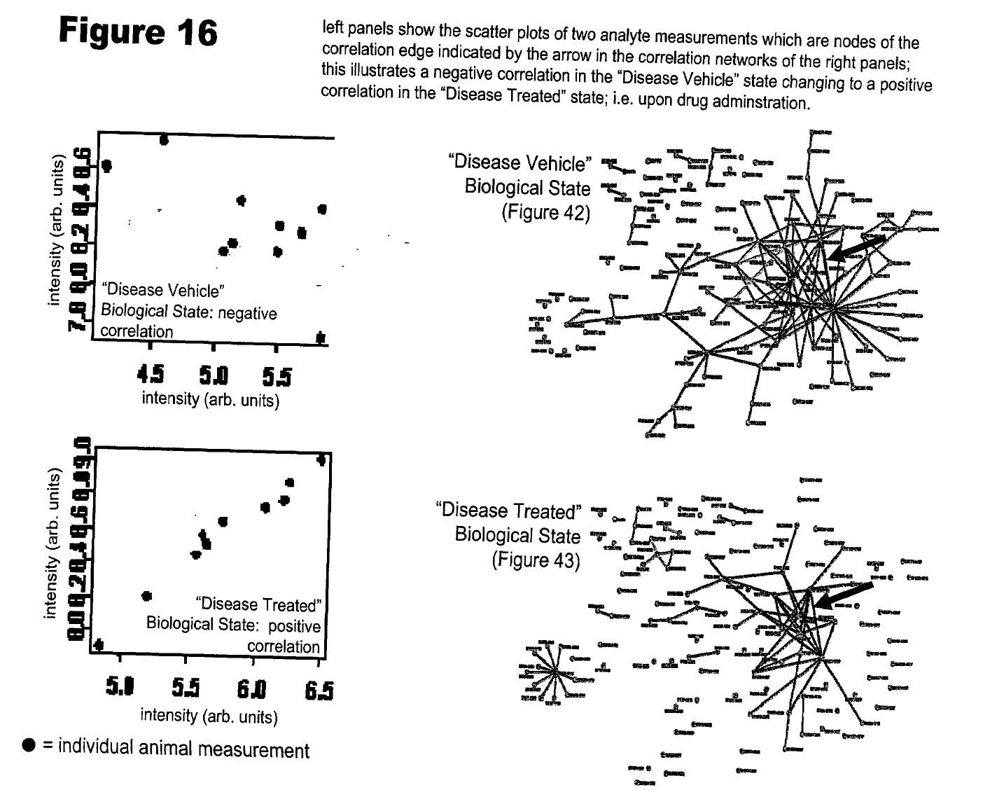

It can be seen from FIGS. 13, 14, and 15 that each of the biological states has a characteristic correlation profile. In addition to these graphical representations, correlations can be listed in a tabular format by listing each possible pair of nodes and the correlation value between them for a given state. Further, it can seen by comparing FIG. 13 and FIG. 14 that the disease has the effect of reversing many correlations which existed in the healthy state, while comparing FIGS. 14 and 15 reveals that intervention by the drug has the effect of partially restoring the correlations altered by disease. FIG. 16 explicitly shows scatter plots of the relative abundance levels of two selected nodes and the corresponding edge from the correlation networks of FIGS. 14 and 15, in order to illustrate the change in correlation in that particular edge between the “Disease Vehicle” biological state and the “Disease Treated” biological state, i.e. the effect of drug treatment upon this aspect of the biological system. It can also be seen by comparing FIGS. 14 and 15 that drug administration also establishes correlations between pairs of analytes where there were none in the health state; these may be indicative of side effects of the drug, side effects being defined as perturbations which do not serve to revert the disease treated state wholly to the control state.

### Data Mining in Correlation Networks: Clustering Coefficient

It can seen from FIG. 12 that correlation networks may contain quite a large number of nodes and edges forming a complex network. One of the objectives of the current study is to discover novel insights into the etiology of the disease as well as the mechanism and effect of the drug. One way to accomplish this objective is to explore the topological and mathematical structure of correlation networks. One such method is to calculate the clustering coefficient of each node in the network, using the following equation:

$${C}_{i} = \frac{{E}_{i}}{{k}_{i} ( {k}_{i} - 1 ) / 2}$$

where Cᵢ is the clustering coefficient of node i, Eᵢ is the number of edges emanating from node i, kᵢ(kᵢ−1)/2 is the total possible edges which could emanate from node i (Watts, D. J. and Strogatz, S. H., “Collective dynamics of ‘small-world’ networks,” Nature, 393: 440-442 (1998)).

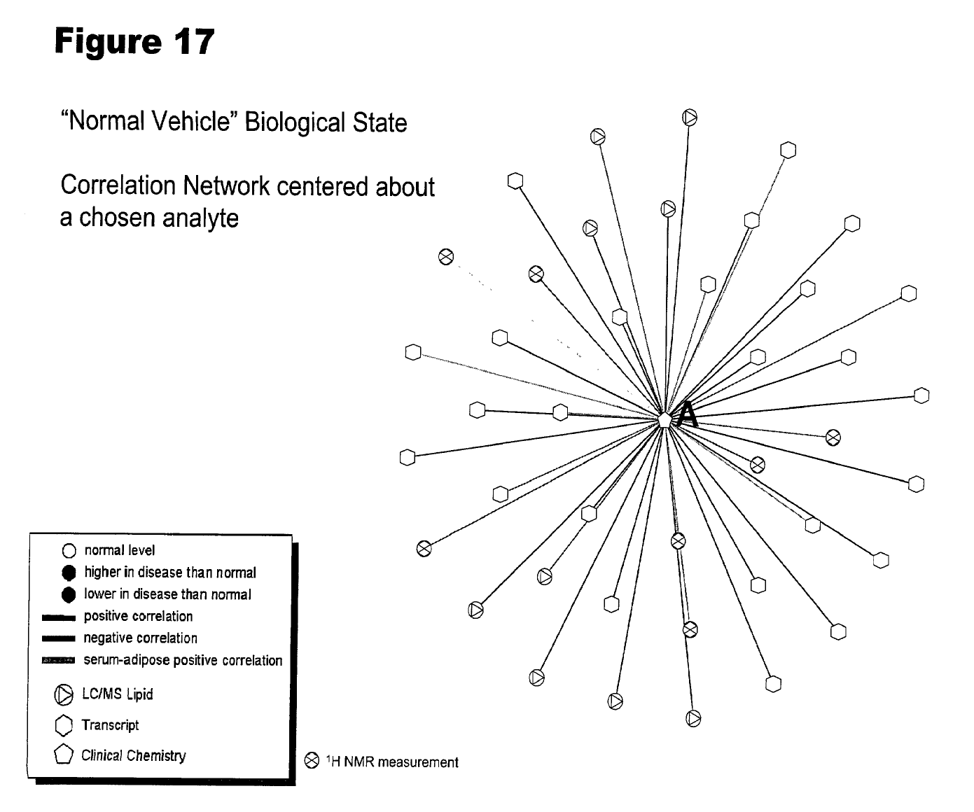

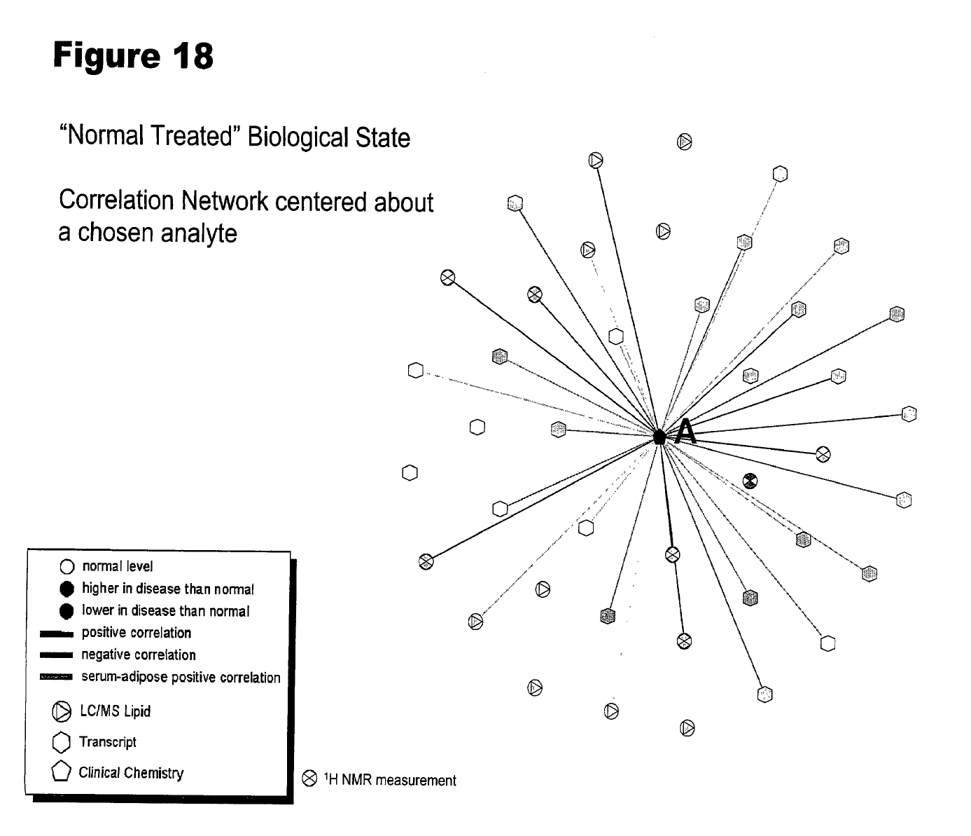

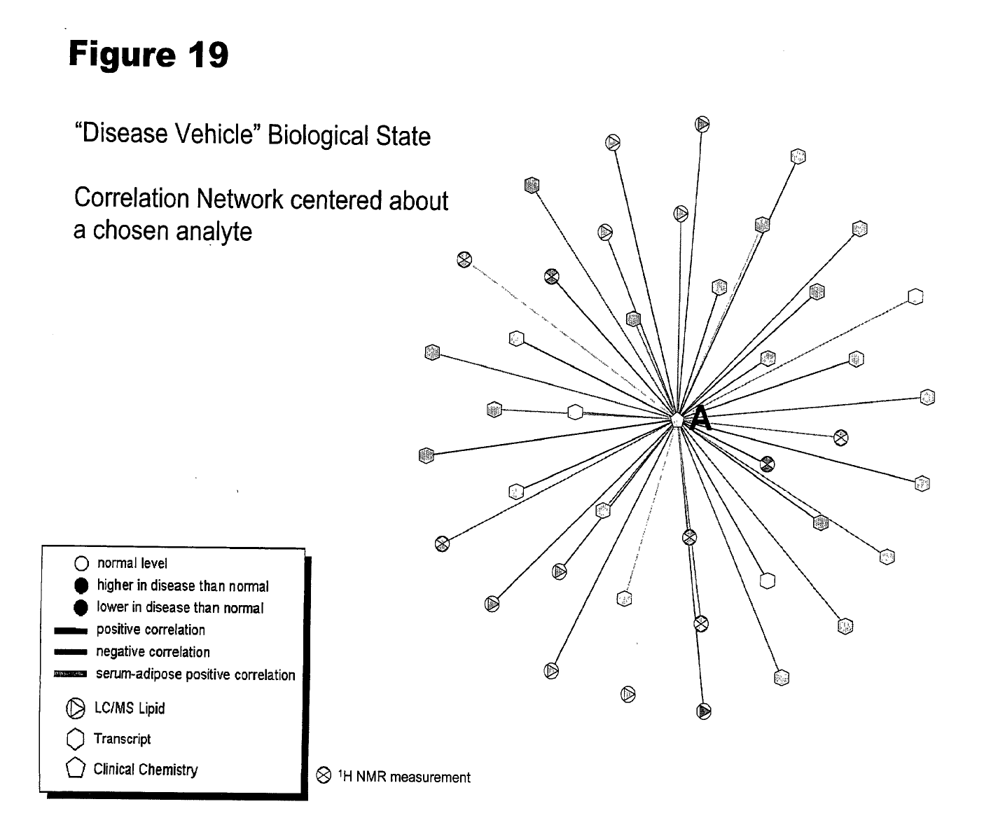

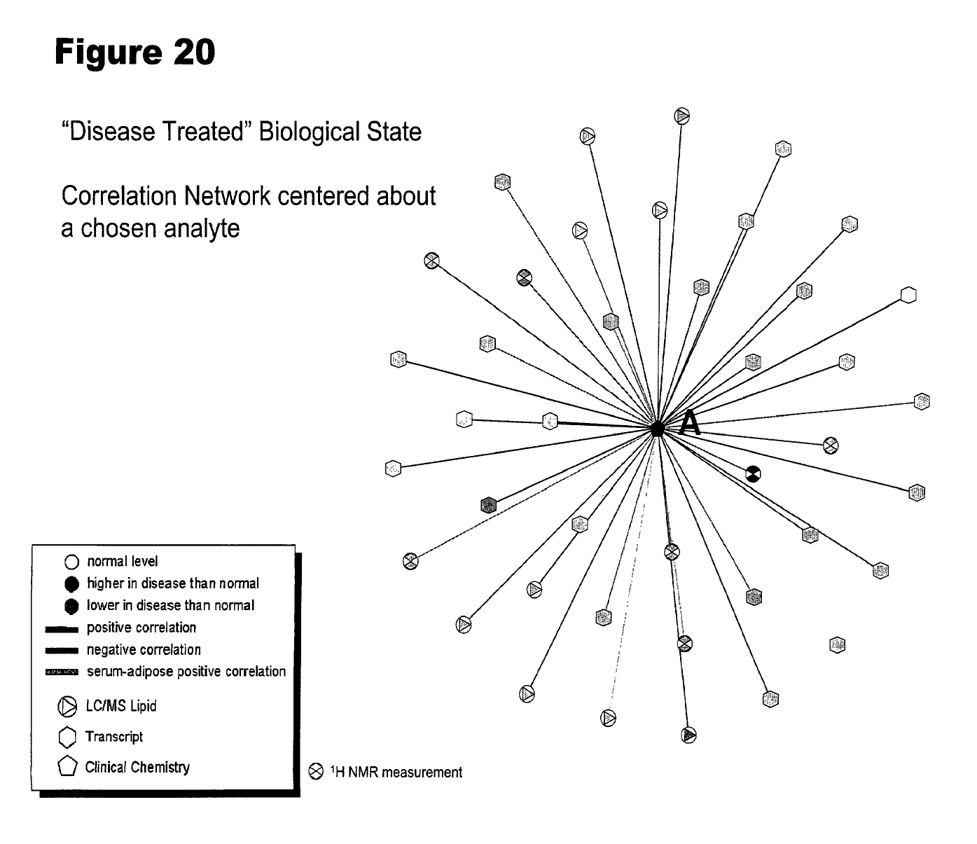

Using this approach, one node (denoted “A”) with a high clustering coefficient which was found in all four biological states in adipose tissue is shown in FIGS. 17, 18, 19, and 20. While this data mining by clustering coefficient is an unsupervised approach in that no a priori biological knowledge about the system is used to pre-select the node(s) of interest, the node “A” selected in this manner was seen to be highly positively or negatively correlated to a number of measurements, as shown in FIGS. 17, 18, 19, and 20, which are highly relevant to the disease etiology under study. FIGS. 17, 18, 19, and 20 show those neighbouring analytes to “A” which are “one-step” away from “A” and which are correlated to A in at least one of the biological states.

Indeed, analyte “A” had hitherto been unappreciated as an important biomolecular analyte in this disease, and the effect of the drug on this analyte had similarly been unappreciated. As has been described above, this node “A” is now prioritized for further exploration and further rounds of experimentation to discern its role in this disease and the effects of this compound; “A” may potentially be a novel drug target or diagnostic or prognostic analyte as it appears to be tightly coupled to other analytes known from prior research by the life sciences community to be important in the etiology of this disease. Indeed, these correlation sub-networks around “A” in adipose tissue revealed a number of interesting analytes corresponding to cell mobility and cell adhesion. To investigate this further, the larger within-state correlation sub-networks, one of which is FIG. 17, were reduced by including only those fat tissue transcripts which are known to be associated with cell mobility and cell adhesion as classified by the Biological Process hierarchy of Gene Ontology.

As mentioned before, FIGS. 17, 18, 19, and 20 are graphical representations of correlation networks centered around node “A”. Indeed, these correlations can also be represented in a tabular format. An example of such a tabular format is shown below.

| | Median- | Median- | | | |
|---|---|---|---|---|---|
| | fold- | fold- | | | |
| | change in | change in | | | |
| | DV state | DR state | | | |
| | vs. NV | vs. DV | | | |
| | state | state | | | |
| Analyte | (“disease | (“drug | r-value in | r-value in | r-value in |
| Symbol | effect”) | effect”) | NV state | DV state | DR state |
| 1115G12 | 1.33 | 2.15 | 0.14 | 0.23 | 0.40 |
| 1208D14 | 2.48 | 1.17 | 0.59 | 0.23 | −0.17 |
| 12041330 | 0.27 | 1.62 | 0.89 | −0.61 | 0.10 |
| 12261460 | 0.42 | 2.69 | 0.73 | 0.00 | 0.83 |
| 12691550 | 0.59 | 2.04 | 0.66 | −0.17 | 0.83 |
| 12701550 | 0.52 | 2.57 | 0.74 | 0.14 | 0.87 |
| 160020121 | 0.70 | 2.28 | −0.12 | 0.80 | 0.31 |
| 217834244 | 0.36 | 2.03 | 0.03 | 0.58 | 0.56 |
| 00037H0 | 0.55 | 2.88 | −0.13 | 0.21 | 0.49 |
| 18045K1 | 2.07 | 0.94 | 0.02 | −0.22 | −0.18 |
| 23106C1 | 0.40 | 0.66 | −0.64 | 0.43 | −0.84 |
| 23042N0 | 0.50 | 2.66 | −0.22 | 0.43 | 0.57 |
| 23146G | 1.71 | 0.45 | 0.22 | −0.81 | −0.33 |
| 241003Bk | 2.26 | 1.03 | 0.66 | −0.40 | 0.43 |
| 28422B0 | 0.73 | 2.24 | −0.02 | 0.87 | 0.25 |
| 405435g565 | 0.31 | 2.19 | 0.55 | 0.00 | 0.84 |
| 493570C0 | 2.00 | 0.97 | 0.48 | 0.55 | 0.25 |
| 49754353 | 0.30 | 1.80 | −0.09 | 0.12 | 0.55 |
| 67208463E0 | 2.12 | 1.33 | 0.56 | 0.50 | 0.43 |
| 6861579 | 0.46 | 1.87 | 0.36 | 0.52 | 0.80 |
| 6921566 | 0.79 | 2.59 | 0.85 | −0.03 | 0.29 |
| 6981683 | 1.59 | 2.08 | 0.59 | −0.12 | 0.92 |
| 7071608 | 0.34 | 2.15 | 0.45 | 0.00 | 0.91 |
| 7331631 | 0.33 | 2.35 | 0.47 | 0.16 | 0.81 |
| 7741403 | 0.36 | 1.39 | −0.07 | −0.31 | 0.81 |
| 7881445 | 0.48 | 1.07 | −0.21 | 0.12 | 0.88 |
| 7931383 | 0.47 | 2.00 | 0.18 | 0.13 | 0.85 |
| 8021255 | 0.69 | 4.15 | −0.07 | 0.40 | 0.93 |
| 8211707 | 2.14 | 1.36 | 0.79 | 0.32 | 0.86 |
| 8321318 | 0.40 | 3.24 | 0.10 | 0.32 | 0.80 |
| 9182094 | 0.33 | 1.01 | −0.89 | 0.06 | −0.53 |
| 9821934 | 0.36 | 0.89 | −0.66 | −0.17 | −0.13 |

### Data Mining in Correlation Networks: Using a Priori Knowledge for Data Mining

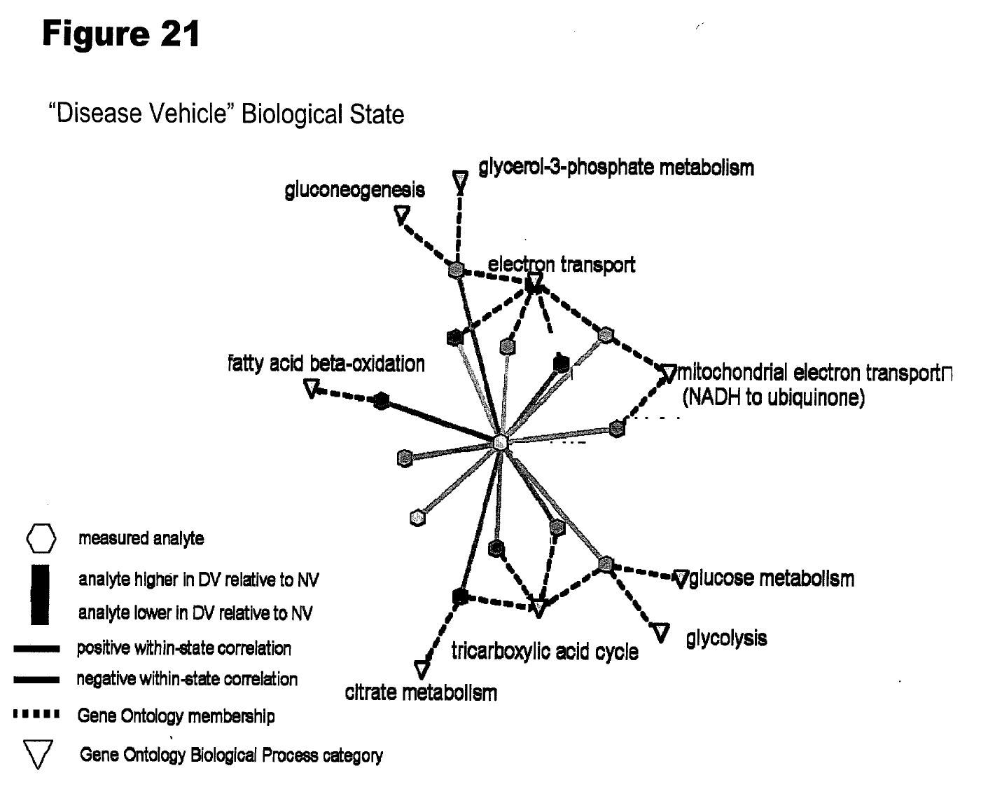

In this current experiment, the researchers are not unknowledgeable of existing scientific literature on this disease state or the effects of the drug under study. An important use of correlation analyses is to focus on measurements of analytes which are a priori implicated in disease etiology or drug effect, both to test existing knowledge and to develop potentially novel hypotheses about disease etiology or drug effect. In the current study, this was done as follows. One source of a priori knowledge of biological analytes is Gene Ontology (www.geneontology.org, November 2004 version). FIG. 21 shows a set of nodes and edges chosen from a larger correlation network (like exemplary FIG. 12) by mapping analytes from the larger network to the Gene Ontology Biological Process hierarchy and subsequently querying for analytes which belong to the biological processes of gluconeogenesis, glycerol-3-phosphate metabolism, electron transport, mitochondrial electron transport, glucose metabolism, glycolysis, tricarboxylic acid cycle, citrate metabolism, and fatty acid beta-oxidation. The analytes which met these criteria, and were also statistically correlated by a positive or negative correlation within the DV state, were then displayed for further exploration.

Indeed, this methodology is not limited to Gene Ontology, but can also be used to create filters to apply to correlation networks based on literature co-occurrence of terms known biochemical pathways such as KEGG (Kanehisa M, Goto S, Kawashima S, Nakaya A., The KEGG databases at GenomeNet, Nucleic Acids Res, 30:42-6 (2002)), and any other a priori data source. When so applied, this approach enriches the correlation network with a priori knowledge, and will provide insight into explaining why certain analytes may be statistically positively or negatively correlated, or may lead to new hypotheses about the roles of analytes whose function in the biological system had hitherto not been known or had been poorly studied.

### Data Mining in Correlation Networks: Cross-Tissue Correlations

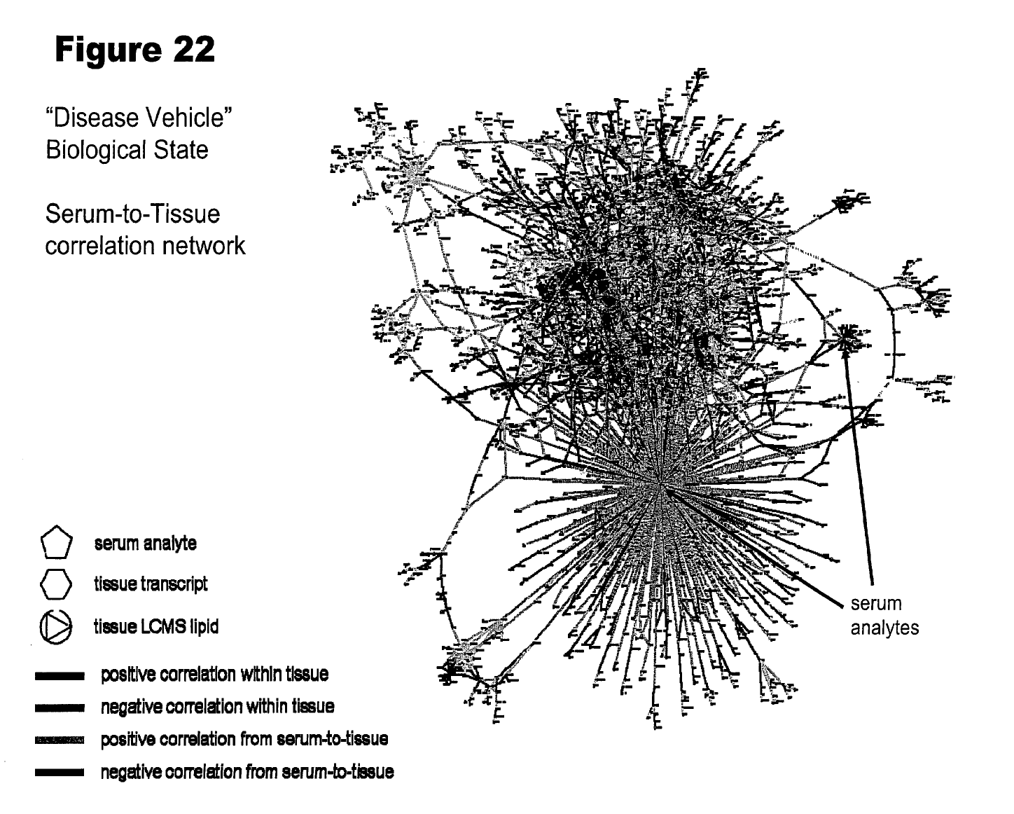

Another important use of correlation analysis is to elucidate relationships between measurements which are made in different biological tissues. In the present example, the two tissues under study are adipose tissue and blood tissue (namely, the serum component of blood tissue). FIG. 22 is one such cross-tissue correlation network. In this network, |r|≧0.9 for each correlation edge. The correlation network in FIG. 22 was constructed using only ten animals in the “disease vehicle” biological state. While much work in the field has been done in attempting to detect certain targeted analytes such as proteins which are presumed to be shed or secreted from one tissue to another, the correlation network approach can be used as an unsupervised survey mode to search for analytes in serum, an accessible body fluid, which are reflective, by virtue of correlation, of biochemical processes occurring in tissue.

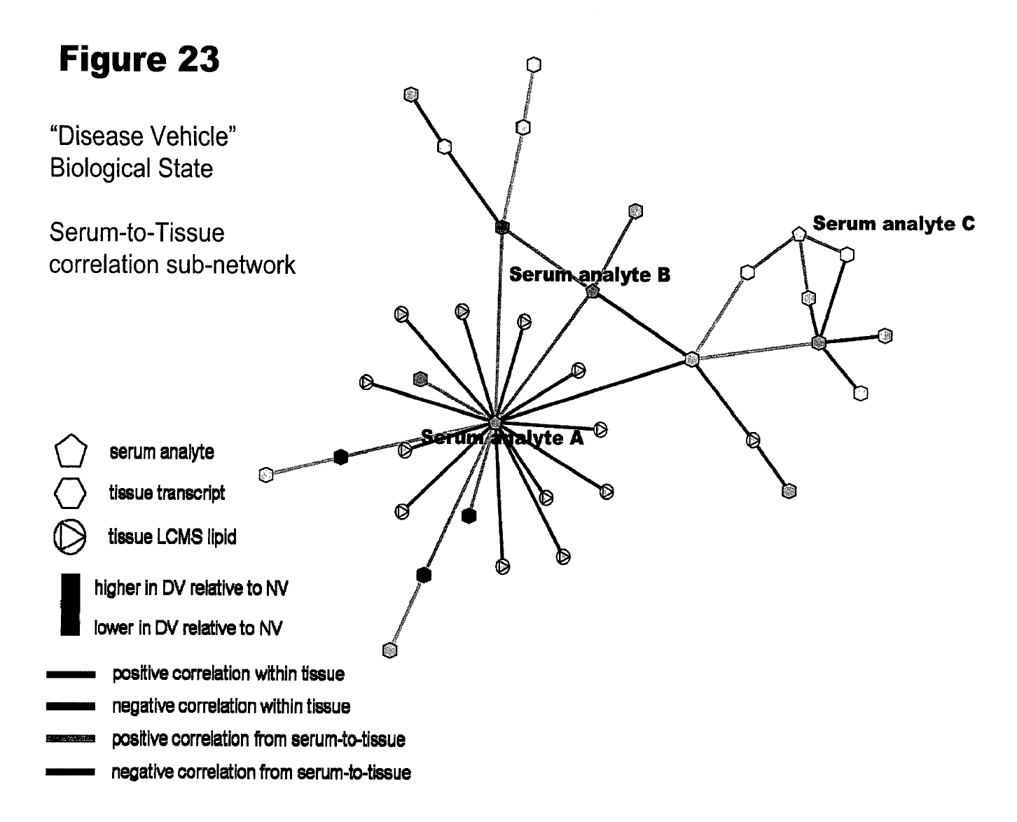

The network of FIG. 22 was further filtered to produce FIG. 23, a smaller network focusing on three serum analytes and the tissue analytes to which they are correlated. The filtering was accomplished by keeping only those tissue analytes which are at most one correlation link away from a serum analyte. It is observed that in this subnetwork a number of tissue mRNA (transcript) measurements and tissue LC-MS lipid measurements are directly correlated with circulating serum analytes which are measured. It is particularly interesting the “Serum Analyte A”, which is higher in abundance in the disease state compared to the healthy state, is correlated to a number of tissue lipids which are, in contrast, lower in abundance in the disease state compared to the healthy state. This may indicate, for example, a negative feedback loop between the serum analyte and these lipids in tissue, or an enzyme whose role it is to maintain levels of these lipids at a certain level in the tissue. In any case, FIG. 23 provides evidence that “Serum Analyte A” is a good surrogate biomarker for the mechanism in which the tissue lipids to which it is correlated are involved.

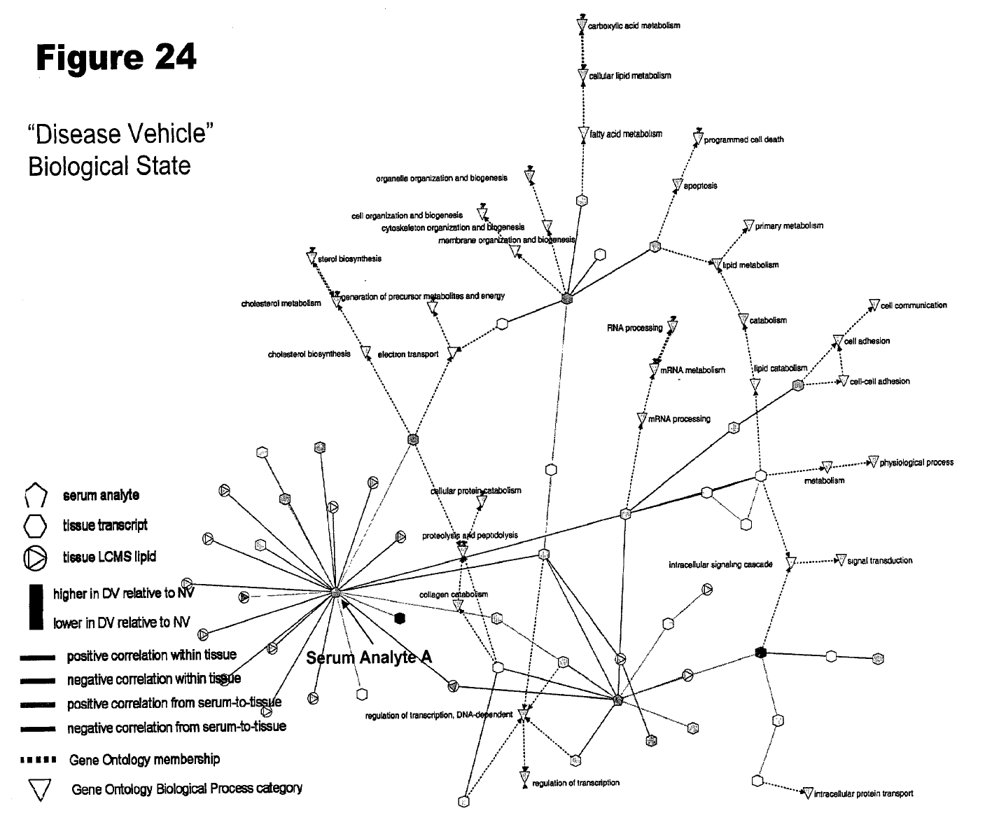

Similar to the approach described above, the subnetwork of FIG. 23 was placed in the context of existing biological knowledge, in this case as represented by Gene Ontology [www.geneontology.org, November 2004 version]. FIG. 24 shows a set of nodes and edges beginning with the correlation network of FIG. 23 and supplemented by mapping analytes in FIG. 23 to the Gene Ontology Biological Process hierarchy. In this way, it was determined that “Serum Analyte A”, for example, was directly correlated to a tissue analyte involved in regulation of transcription, and another tissue analyte involved in cholesterol biosynthesis and cholesterol metabolism. As such, “Serum Analyte A” may be hypothesized to be a hitherto unappreciated surrogate biomarker of a number of important aspects of disease etiology in the current study, including regulation of transcription, cellular protein catabolism, sterol biosynthesis, carboxylic acid metabolism, programmed cell death, signal transduction, and other processes reflected in FIG. 24.

As stated before, this methodology is not limited to Gene Ontology, but can also be used to create filters to apply to correlation networks based on literature co-occurrence of terms known biochemical pathways such as KEGG, and any other a priori data source.

### Correlation Analysis of Rat Model of Drug-Induced Toxicity

### Selecting Body Fluid Biomarkers for Drug-Induced Toxicity of the Liver

The study focused on the application of systems biology approaches to the discovery and characterization of biomolecular markers (biomarkers) associated with liver steatosis induced by a pharmaceutical compound, ABC123. The primary objective of the study was to discover biomarkers in plasma of hepatic steatotic processes. In the course of this study, multiple molecular profiling techniques and data analysis methodologies were employed. A corollary objective of this study was to elucidate mechanisms underlying hepatic steatosis induced by the drug.

The key hypothesis upon which the study was based is that there are molecular species, beyond those identified by standard histological techniques or clinical chemistry measures, which can be found in liver, plasma or urine samples from male Wistar Hannover rats to discriminate between drug exposures that cause liver steatosis and those exposures that do not induce steatosis. Furthermore, by analyzing and integrating the proteomic, metabolomic and transcriptomic profiling data derived from control rats vs. drug-treated rats using an integrative systems biology approach, one can identify such molecular biomarkers, particularly in easily accessible body fluids such as plasma, which are reflective of drug-induced toxicological processes manifest in the liver organ of this mammalian model.

### Study Design

The study was designed to generate tissue and body fluid samples from groups of animals exposed for varying times at different doses to a drug previously shown to produce toxic steatosis of the liver.

Serum, urine, and liver samples from control male rats and from male rats exposed to drug ABC123, a known inducer of hepatic steatosis, were collected. Five Wistar Hannover male rats were used per group. Three groups were dosed for 7 days (control, low and intermediate dose) and urine collected twice daily for up to 7 days. One group was dosed once with a high dose of the drug and kept in a metabolism cage for a recovery study. In addition, three groups (control, intermediate and high dose) were dosed once and sacrificed after 24 hours. Plasma and liver tissue samples were collected from all groups at necropsy. Clinical pathology was performed and histopathological evaluations were provided.

| | Animals | | Dosing | | | | |
|---|---|---|---|---|---|---|---|
| | (number | Dosage | Period | Sacrifice | Available Samples | | |
| Group Label | & sex) | (mg/kg/day) | (days) | Day | Liver | Plasma | Urine |
| 1 | 5 M | 0 | 7 | 8 | ✓ | ✓ | ✓ |
| 2 | 5 M | 20 | 7 | 8 | ✓ | ✓ | |
| 3 | 5 M | 200 | 7 | 8 | ✓ | ✓ | ✓ |
| 6 | 5 M | 200 | Single dose | 2 | ✓ | ✓ | |

Animal groups in hepatotoxicity study and available samples.

The study was designed to generate tissue and body fluid samples from groups of animals treated for varying times and with different doses of ABC123. The different time points and dosing schedules were intended to allow the identification of biomarkers of steatosis over different times and doses.

It should be noted that Group 3, the group of rats that had received the highest cumulative dose, was the only group to reveal morphological steatosis upon examination of the livers using standard morphology techniques. Animals subjected to the lowest dose (Group 2) showed no evidence of steatosis, thus precluding the study of dose effect.

Groups 1, 2, 3, and 4 were necropsied on day 8, Groups 5, 6, and 7 were necropsied on day 2 and Urine samples were available for the following 6 time points: predose, days 1, 2, 3, 5, and 7.

### Analytical Profiling of Samples

The different biological samples and analytical platforms used in this study were as follows:

Metabolites and Proteins
Plasma (LC-MS lipid, GC-MS)
Urine (NMR, GC-MS)
Liver (LC-MS lipid, LC-MS proteomics)

Transcripts from liver tissue

Liver and plasma lipid LC-MS. Samples (liver or plasma) were treated with isopropanol to precipitate the protein and to extract the lipid metabolites. The isopropanol contained three reference standard compounds. Samples were vortexed to mix, centrifuged and the supernatant removed for analysis. The set of isopropanol extract fractions corresponding to the set of samples were loaded into a Waters 717 auto sampler and separated on a Waters 600-MS HPLC system employing a C4 column at 1 mL/min with a gradient from 5% methanol/water to 100% methanol with both containing 10 mM NH₄Ac and 0.1% formic acid. The output of the HPLC was connected to a Finnigan TSQ 700/7000 equipped with electrospray for MS and MS/MS analysis. Resulting mass spectra were peak detected with IMPRESS (proprietary software, BG Medicine, Inc., Waltham, Mass.) and aligned/normalized with Equest and WinLin (proprietary software, BG Medicine, Inc., Waltham, Mass.). The three internal standards mixed with the samples ensured accurate alignment and normalization. After alignment and normalization the dataset of spectral peaks for all samples in the LC-MS run was processed by a number of mathematical approaches to identify univariate and multivariate biomarkers (see appropriate methods section). Metabolites detected with this approach include polar and non-polar lipids.
Plasma and urine GC-MS. Urine samples were freeze-dried and plasma samples were extracted with methanol and dried under nitrogen. After this first step was complete, both sample types were derivatized with oximation and subsequently silylated. The derivatized samples were loaded in an ATAS Focus autosampler and separated on an Agilent 6890 gas chromatograph. The samples were detected with electron impact ionization on an Agilent 5973 MSD. Six internal standards were employed in this workflow. Subsequent to detection, the samples were processed in the same manner as the liver and plasma lipids. Metabolites detected with this method include: alcohols, aldehydes and cyclohexanols, amino acids, acyl amino acids, succinylamino acids, amines, aromatic compounds, fatty acids (>C6), organic acids, phospho-organic acids, sugars, sugar acids, sugar amines, and sugar phosphates.
Urine NMR. Typical metabolites detected with this approach include: amino acids, organic acids and sugars. Urine samples were lyophilized and dissolved in a sodium phosphate buffer at pH 6.0 in D₂O. In this study, 1D urine NMR spectra were acquired on a Bruker AVANCE spectrometer operating at 600.13 MHz ¹H resonance frequency. Even at this high frequency, 1D ¹H spectra of biological fluids such as urine still show considerable peak overlap in certain chemical shift ranges (especially the ‘aliphatic’ region of the spectrum from δ 0.8 to 4.5), that have in earlier days been described in terms of chemical noise. This chemical noise occurs where there is multiple overlap and superposition of peaks arising from low concentrations of metabolites that are within the NMR detection range (Foxall P, Parkinson J, Sadler I, Lindon J, Nicholson J., Analysis of biological fluids using 600 MHz proton NMR spectroscopy: application of homonuclear two-dimensional J-resolved spectroscopy to urine and blood plasma for spectral simplification and assignment, J Pharm Biomed Anal. 11(1):21-31 (1993)). The data was processed with Bruker software as well as WinLin (proprietary software, BG Medicine, Inc., Waltham, Mass.). NMR peaks relevant to differences between the groups of data studied were assigned via comparison against both literature references and authentic reference standards using an in-house spectral data-base. Additional confirmation of assignments was made by the application of two-dimensional (2D) NMR methods; i.e., 2D TOCSY and 2D J-resolved (JRES). The assignment of signals was complicated both by the difference in pH of the rat urine samples (pH adjusted to 6.0) and the reference standards (pH range of 7.0-7.4) and by the chemical noise in the spectra.

### Liver Protein Analysis.

Overview of Proteomics Solid Tissue Liver Platform Workflow in this Study:

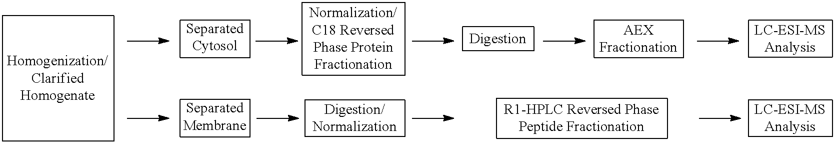

where AEX refers to anion exchange; HPLC refers to high performance liquid chromatography; and LC-ESI-MS refers to liquid chromatography-electrospray-mass spectrometry).

### Laboratory Methods:

Sample: 100 mg of liver dissected from the thawed liver sample was cut into four 25 mg pieces and homogenized.
Clarification/Fractionation: Unbroken cells, nuclei and extracellular debris were removed using low-G centrifugation, and the resulting clarified homogenate was then subjected to membrane/cytosol fractionation using high-speed ultracentrifugation.
Protein Fractionation of Cytosol: The total cytosolic protein was subjected to C4 reversed-phase column chromatography to isolate 3 fractions for proteomic analysis. The column was cleaned between each sample run with a high concentration of formic acid to remove remaining bound material; this fraction was not analyzed.
Acidic Peptide Selection: Each of the three protein cytosolic fractions from the prior step was trypsin digested and from each fraction the resulting three acidic peptide fractions (generally those containing at least 2 aspartate/glutamate residues) were isolated via AEX, and desalted by reversed-phase column chromatography prior to LC-ESI-MS analysis.
Membrane Digest Isolation: Membrane fraction proteins were also trypsin digested. Digestion reagents and undigested and partially digested materials were separated from the tryptic peptide fraction by R1-C18 reversed-phase HPLC chromatography and discarded. The resulting membrane tryptic peptide fraction was dried in vacuo.
Profiling of Peptides: Cytosolic peptides from the three acidic peptide sets and the membrane tryptic peptide fraction were quantitatively analyzed for differential expression via reversed-phase LC-ESI-MS. The HPLC gradient was designed to provide reproducible retention times (RT) for the corresponding eluting peptides from one sample to the next. For this profiling step, the mass spectrometer was configured to measure eluting peptide mass to charge ratios (m/z) with high mass accuracy and resolution.
Peak Identification and Data Alignment: GISTools™ software (proprietary software, BG Medicine, Inc., Waltham, Mass. re) is used for peak picking. It characterizes peptide LC-MS data by m/z, retention time, isotope, charge and intensity. Scanfinder software (proprietary software, BG Medicine, Inc., Waltham, Mass.) was used to align peptide signals from multiple GISTools™ peak tables.

### Peptide Identification

Peptide MS/MS spectra were acquired during LC-MS/MS acquisition runs that were interspersed between LC-MS profiling acquisitions at a frequency of approximately one MS/MS acquisition per three MS runs. Spectra were converted to SEQUEST style DTA files using the MassLynx PeptideAuto post processing program. DTA files are an ASCII format encoding the precursor MH+ and charge along with a listing of fragment ion m/z and abundances. Separate in-house created software maps MassLynx scan/function identifiers associated with each spectrum to LC retention times. After uploading LC-MS/MS data and DTAs into BG's LIMS system, an automated processing pipeline launches SEQUEST and Mascot search algorithms. Additional searches using X!Tandem were performed on an ad-hoc basis. SEQUEST and Mascot search results were uploaded into BG Medicine's LIMS system and an in-house program, PTCruiser, was used by skilled artisans in the identification of peptides by MS/MS.

Briefly, spectra were grouped by the peptide sequence models proposed by the searching algorithm and peptides were grouped by protein. PTCruiser is the web interface that skilled artisans can use to view spectra in the context of the search algorithm proposed peptide sequence models, view spectra from the same peptide that were previously validated, view alternative models proposed for the same spectra and to capture their comments after their analysis. Spectra were reviewed for the quality of the peptide model ultimately deciding if they felt the proposed peptide sequence was correct with high confidence. High confidence models were recorded as “validated” into the database. These validated peptides were subsequently cross checked for agreement between the 3 independent search algorithms (SEQUEST, Mascot and X!Tandem) and mass accuracy subsequent to a boot-strapped recalibration procedure. This boot-strapping recalibration procedure calculates a median PPM offset per LC-MS/MS run from spectra within that run where a.) the search algorithm proposed peptides that were both previously validated in BG Medicine's peptide spectral library and b.) the spectrum passed an initial filter based on SEQUEST XCorr. The calculated median offset was then applied to every spectrum acquired in that particular LC-MS/MS acquisition run.

MS/MS spectra were matched to peaks in the profiling alignments in a manner analogous to that used to create the profiling alignments except that the boot strapping recalibration procedure was used to increase m/z precision and accuracy and that the observed ranged of retention times for the set of peaks in an “aligned peak” were used as the basis for matching to the recalibrated m/z and retention time of MS/MS spectra.

### Peptide to Protein Mapping

To maximize our ability to interpret protein isoforms in the proper biological context we validated the mapping of peptides into proteins using a set theoretic approach with the Protein Validation Tool (PVT™). The input to this process is the set of peptide sequences derived from Sequest, Mascot, PTCruiser™ and skilled artisan validation; and the PIR-NREF protein sequence database version 1.40 (Wu C. H. et al., “The Protein Information Resource,” Nucleic Acids Research 31(1):345-347 (2003)), filtered for mammalian sequences. The output of PVT™ is a map fitting all peptides into their protein instances, and a map of all protein instances into their protein class (a “protein instance” is a protein with a unique string of amino acid residues in a given species. The PIR-NREF protein sequence database is a good example of a protein instance database).

PVT™ takes the set of peptides and searches each sequence against all sequences in the protein sequence database, allowing isoleucine and leucine to substitute for each. Other than this substitution, only perfect matches are permitted; i.e., no mismatches or gapping is allowed. The set of matched protein instances is then ordered by the number of peptides mapped to each. Then each pair of instances is evaluated for their set relationship (equal, disjoint, subset, superset), determining whether two protein instances are part of the same class, are independent or one is contained by another. When multiple protein instances contain the same set of proteins and there is no a priori way of distinguishing the best mapping a protein exemplar is chosen based on the correct species and the longest sequence. Protein classes are then evaluated to determine whether they are too inclusive by comparing the mapping of their instances back to the Rattus norvegicus genome. Protein instances are recorded as PIR-NREF identifiers while protein classes are recorded as Locuslink identifiers.

Transcript (mRNA) Profiling

Affymetrix microarray processing was carried out on liver tissue samples from 35 animals distributed among the seven experimental groups. The Affymetrix U34A chip was used (Affymetrix U34A chip, version December 2003) for all hybridizations.

Beginning with the *.cel files, the data were quantile normalized, and positional-dependent nearest-neighbor (PDNN) output from the quantile normalized *.cel files were computed (Zhang L., Miles M. F., Aldape K. D., “A model of molecular interactions on short oligonucleotide microarrays,”. Nat Biotechnol. July 21(7):818-21 (2003)). An additive effect on the log-transformed signal related to hybridization day was modeled, and the triplicate control samples were used to estimate the effect. A significant hybridization day effect was observed and compensated for in the analysis by the removal of the estimated additive hybridization day effect per gene. Subsequently, differential expression analysis was preformed using the Significance Analysis of Microarrays (SAM) approach of Tibshirani et al., with a false discovery rate of 7% (Tusher V. G., Tibshirani R., Chu G., “Significance analysis of microarrays applied to the ionizing radiation response,” Proc. Natl. Acad. Sci. USA 98(9):5116-21 (2001)). The PDNN algorithm responded well to removal of hybridization day effect in PCA analysis, and the SAM analysis yielded large gene lists for some group comparisons, smaller lists for others.

### Univariate Data Analysis

Plasma and liver biomarkers for exposure to ABC123 and candidate biomarkers for toxicological effects in liver were obtained following within-platform analysis of variance (ANOVA). The ANOVA model is a generalization of the well known t-test setting, in which more than two groups are tested for changes (shifts) in means. In this study, the different treatment dose and duration combinations gave rise to seven treatment groups, namely groups 1, 2, 3, 4, 5, 6 and 7. Every spectral measurement in a dataset was tested individually and was declared a marker of animal exposure to the drug if the measurement (or analyte) had statistically significant differences in level of expression between at least two treatment groups in the study. Furthermore, each of the marker peaks was tested for a family of four specific pair wise group comparisons that were deemed to be scientifically interesting, namely Group 3 vs. Group 1, Group 3 vs. Group 2, Group 2 vs. Group 1, and Group 6 vs. Group 1. Markers that showed differences in the Group 3 vs. Group 1 comparison differentiate animals that received the highest exposure to the drug from the control animals. On the other hand, markers that showed statistically significant differences in the Group 2 vs. Group 1 comparison can be considered early biomarkers of animal exposure to the drug and candidate early biomarkers of hepatotoxicity.

Because urine samples were collected at multiple time points on days 0, 1, 2, 3, 5 and 7 from all animals in Groups 3 and 1, the analysis of this data involved fitting a Repeated Measures ANOVA model. This model is a generalization of the ANOVA model to take into account longitudinal data structures, where linear, quadratic and cubic effects of time on the mean value of the analyte were permitted. Every analyte was tested individually and was declared a marker if a test of equality of the group mean profiles between Groups 1 and 3 was rejected.

Every analyte measured in the various platforms has an associated p value derived from the univariate ANOVA tests described above. When only a single statistical test is performed with a significance value α decided a priori (typically α=5% or 10%), would the null hypothesis be rejected and the test declared to be significant at level α if the p-value of the test statistic is smaller than α. However, when multiple tests are performed, a few small p-values by random chance can be expected. To illustrate this point, consider the following example: if 1000 t-tests are performed on 1000 different analytes and if none of the 1000 different analytes is in reality differentially expressed, about 50 p-values lower than 5% would be expected. Thus, if all the tests with p-values lower than 5% were declared to be significant, about 50 false discoveries (and a false discovery rate of 100%) would likely result. In this study, each platform yielded a rich set of several thousand analytes to work with and hence this issue of multiple testing was very relevant in the analyses. To take this into consideration, the p-value resulting from ANOVA was replaced by the False Discovery Rate-corrected p-value (FDR) (Y. Benjamini and Y. Hochberg, “Controlling the false discovery rate: A practical and powerful approach to multiple testing,” Journal of the Royal Statistical Society (Series B), 57(1):289-300 (1995)), which controls the ratio of the false discoveries to the total number of discoveries. This adjustment ensures that among all analytes with FDR adjusted p-value less than 5% (i.e. all analytes discovered to be biomarkers), the expected proportion of false discoveries is bounded by 5%.

### Correlation Analysis

In this study, because the number of animals per group was relatively low, we chose to focus on correlations or trends between pairs of analytes that persist across treatment groups. The mathematical term for a correlation of this type is “partial correlation” (Blalock, H., “Causal inferences in nonexperimental research,” Chapel Hill, N.C.: UNC Press (1961)). This method involves calculating correlations after group specific means are removed, which allows one to discount spurious associations between two analytes that can appear due to differences in expression levels between treatment groups in either one or both of the analytes considered.

Partial correlations for all pairs of analytes were then used to generate correlation networks. These networks are graph representations of sets of correlations, where nodes or vertices are measured analytes (e.g. gene transcripts, clinical chemistries, lipids, NMR metabolites, proteins etc.) and edges are derived correlations between any pair of analytes. The general approach to constructing a correlation network is to first determine all pairs of correlations among the set of measured analytes, irrespective of tissues and platform types. Inclusion criteria are applied to the putative network to limit its scope to biologically relevant and/or tractable observations. These criteria can include: mean or median fold changes for analytes in a disease model (e.g. mean fold change of wild-type animals without treatment over diseased animals without treatment); false discovery rate (FDR) thresholds on correlations (Ho: ρ=0); ANOVA tests across all treatment groups looking for markers; correlations surrounding a known biological marker or target; and/or other criteria as deemed necessary.

In keeping with our goals of identifying biomarkers in plasma that are reflective of mechanisms in the liver, we generated two sets of correlation networks. The first network presents all pair wise correlations between analytes in liver paired with analytes in plasma (Plasma-Liver Correlation Network). The second network presents all pair wise correlations between analytes within liver (Liver-Liver Correlation Network). To correct for correlations that may be driven by mean separations alone, correlations were calculated across all treatment groups after removing group specific means. Both correlation networks included data from all animals in Groups 1, 2, 3 and 6. The plasma analytes included metabolites measured from both the GC-MS and LC-MS platforms. The liver analytes included transcripts, proteins from cytosolic fractions 1, 2 and 3, proteins from the membrane fraction and metabolites from the LC-MS platform. All liver and plasma analytes that rejected the test of equality of group means with a corresponding FDR p value less than 0.15 were included in the network. In addition, all identified liver peptides were included regardless of their FDR p values. In both networks, two nodes were considered connected by an edge if and only if their corresponding value of correlation met certain threshold criteria, where these criteria were chosen in order to limit the complexity of the resulting graph. The Plasma-Liver and the Liver-Liver networks were combined into a single graph for bioinformatics analyses.

For the purposes of biological interpretation, working at the level of proteins instead of peptides is generally desired. To represent proteins in the correlation network, “protein instance” nodes were inserted into the network and the peptide nodes that map into this protein instance (see PVT section above) were connected with edges of type “part of protein instance.” If all of the peptides that make up a protein instance are either changing in expression in the same direction or are unchanged, then the protein instance will be assigned the expression value of the peptide that exhibits the greatest change in expression. If in the set of peptides that make up the protein instance there are peptides that increase in expression and peptides that decrease in expression, then no expression value will be assigned to the protein instance.

For proteins in correlation networks, correlations between other analytes and proteins, rather than peptides, should be used. Therefore inserted into the network was an “implied correlation” edge between a protein instance and an analyte if at least one of the constituent peptides of the protein instance correlates with the analyte. An insertion is made because a correlation between an analyte and a protein is implied if there is an underlying correlation between the analyte and a peptide that is a part of the protein. If there are several peptides that correlate with the analyte and not all of the correlations are of the same sign (i.e., all positively correlated or all negatively correlated), an implied correlation edge is not inserted. Similar to the assignment of the expression value to protein instance nodes, implied correlation edges are assigned the value of underlying correlation edge that has greatest absolute value.

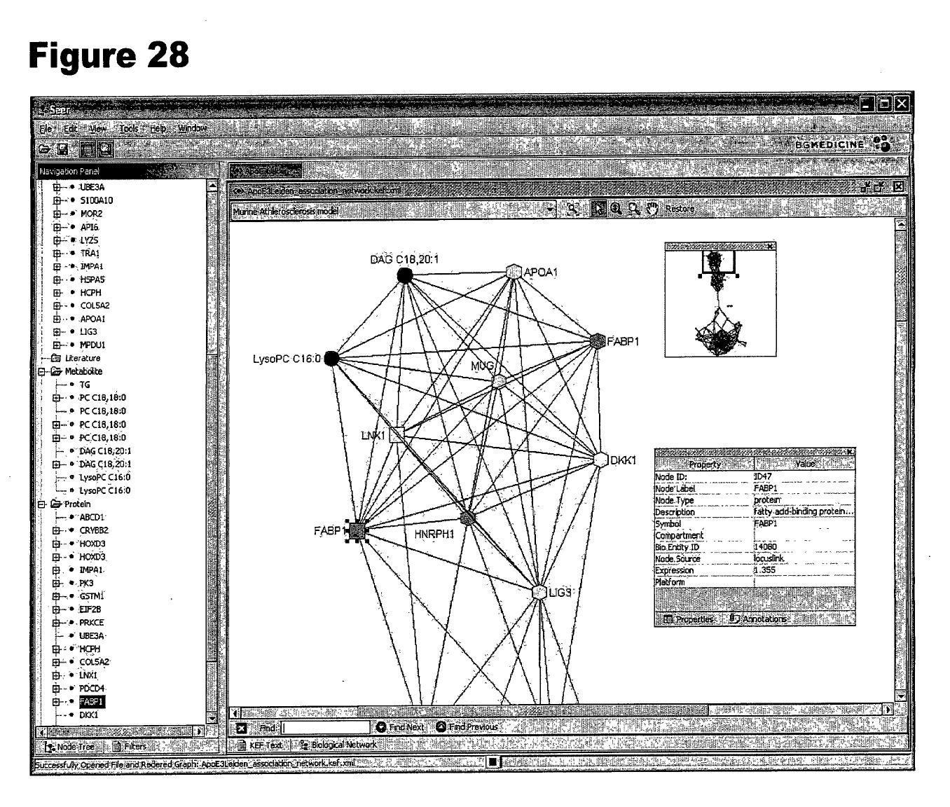

A software program known as Seer™ was used to visualize the correlation networks (see FIG. 28 for a screen shot of Seer). Symbols (called “nodes” in graph theory) represent analytes and their shape indicates the platform used to measure the analyte. Nodes are colored to indicate a change in expression between two states, where each state in this study is a treatment group. A greater red intensity indicates increased expression in the experimental state compared to a reference state. Similarly, a greater green intensity indicates a decreased expression when comparing two states. Lines (called “edges” in graph theory) represent a connection between two nodes, and are used to denote correlations between two analytes. Edges are colored according to the correlation coefficient they represent where a greater red intensity denotes a more positive correlation and a greater green intensity denotes a more negative correlation.

### Univariate Analysis Results

Univariate analysis of each metabolomic and cytosolic proteomic platform revealed many spectral peaks with statistically significant differences in intensity when comparing across all cohorts, generally with the fewest such analytes in the comparison of Groups 1 vs. Group 2, and the most in the comparison of Groups 1 vs. Group 3.

By contrast, univariate analyses of membrane-derived proteolytic peptide profiles revealed statistically significant differences in comparisons of all groups, including many within the comparison of Group 1 vs. Group 2.

Further, longitudinal temporal analyses of features in urine established statistically significant differences as early as the second day of treatment.

Selected results derived from the plasma body fluid compartment are presented below.

### Plasma GC-MS

Univariate analysis of plasma GC-MS analytes resulted in several markers for each of the three group comparisons, 3-1, 3-2, and 2-1, respectively. Specifically, 334, 265, and 115 analytes were found to have FDR adjusted p values less than 0.05 for the group comparisons 3-1, 3-2, and 6-1, respectively. See the table below for details. None of the analytes from this platform was found to act as a marker distinguishing Group 2 from the control animals (Group 1).

### Plasma GC-MS Univariate (ANOVA) Analyses: Number of Spectral Peaks Meeting Statistical Criteria

| Group Comparison | Statistical Criteria | Plasma GC-MS Peaks |
|---|---|---|
| Group 3-Group 1 | p_{FDR} ≦ 0.15 | 540 |
| | p_{FDR} ≦ 0.10 | 520 |
| | p_{FDR} ≦ 0.05 | 334 |
| | p_{FDR} ≦ 0.01 | 135 |
| Group 3-Group 2 | p_{FDR} ≦ 0.15 | 429 |
| | p_{FDR} ≦ 0.10 | 415 |
| | p_{FDR} ≦ 0.05 | 265 |
| | p_{FDR} ≦ 0.01 | 106 |
| Group 2-Group 1 | p_{FDR} ≦ 0.15 | 0 |
| | p_{FDR} ≦ 0.10 | 0 |
| | p_{FDR} ≦ 0.05 | 0 |
| | p_{FDR} ≦ 0.01 | 0 |
| Group 6-Group 1 | p_{FDR} ≦ 0.15 | 128 |
| | p_{FDR} ≦ 0.10 | 126 |
| | p_{FDR} ≦ 0.05 | 115 |
| | p_{FDR} ≦ 0.01 | 76 |

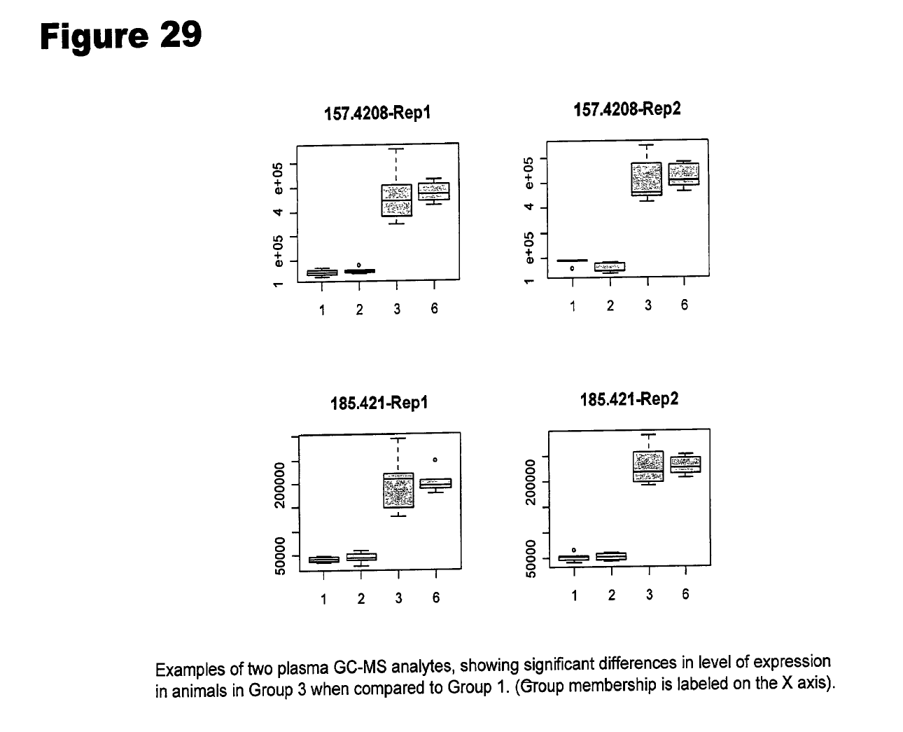

As examples, FIG. 29 shows box plots of the distribution of two analytes, 157.4208 and 185.421, which show highly significant differential expression in Group 3 animals when compared to the control animals (Group 1). Analyte 157.4208 shows median fold change of 7.0, whereas analyte 185.421 shows a median fold change of 5.1 for the Group 3 vs. Group 1 comparison, where median fold change is calculated as the ratio of the median expression in Group 3 to that in Group 1.

### Plasma Lipid LC-MS

Univariate analysis of plasma Lipid LC-MS analytes resulted in 71 and 70 spectral peaks with FDR adjusted p values less than 0.05 for the group comparisons 3-1 and 3-2, respectively. See the table below for details. None of the analytes from this platform was found to act as a marker distinguishing either Group 2 from Group 1, or Group 6 from Group 1.

| | Statistical | Plasma Lipid |
|---|---|---|
| Group Comparison | Criteria | LC-MS Peaks |
| Group 3-Group 1 | p_{FDR} ≦ 0.15 | 73 |
| | p_{FDR} ≦ 0.10 | 73 |
| | p_{FDR} ≦ 0.05 | 71 |
| | p_{FDR} ≦ 0.01 | 33 |
| Group 3-Group 2 | p_{FDR} ≦ 0.15 | 71 |
| | p_{FDR} ≦ 0.10 | 71 |
| | p_{FDR} ≦ 0.05 | 70 |
| | p_{FDR} ≦ 0.01 | 32 |
| Group 2-Group 1 | p_{FDR} ≦ 0.15 | 0 |
| | p_{FDR} ≦ 0.10 | 0 |
| | p_{FDR} ≦ 0.05 | 0 |
| | p_{FDR} ≦ 0.01 | 0 |
| Group 6-Group 1 | p_{FDR} ≦ 0.15 | 0 |
| | p_{FDR} ≦ 0.10 | 0 |
| | p_{FDR} ≦ 0.05 | 0 |
| | p_{FDR} ≦ 0.01 | 0 |

### Plasma Lipid LC-MS Univariate (ANOVA) Analyses: Number of Spectral Peaks Meeting Statistical Criteria

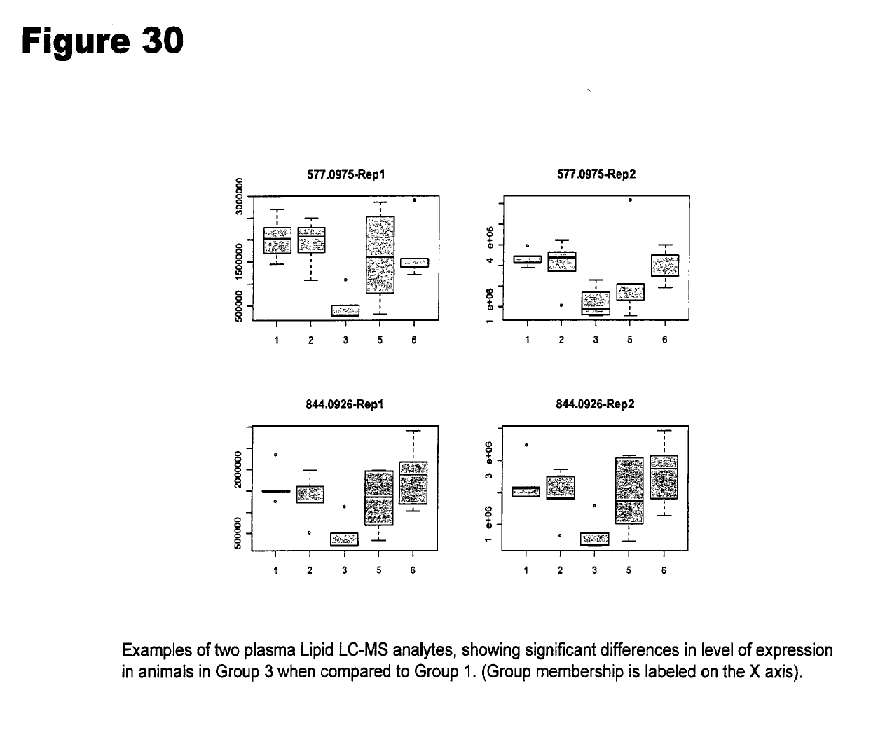

As examples, FIG. 30 shows box plots of the distribution of two analytes, 577.0975 and 844.0926, which show highly significant differential expression in the Group 3 vs. Group 1 comparison. Analyte 577.0975 shows a median fold change of 7.1 whereas analyte 844.0926 shows a median fold change of 7.0 for the Group 3 vs. Group 1 comparison, where median fold change is calculated as the ratio of the median expression in Group 1 to that in Group 3.

In summary, both LC-MS and GC-MS platforms on plasma samples yielded several strong biomarkers serving to differentiate the extreme groups, namely animals in Group 3 versus control animals (Group 1). The analytes found to differentiate animals in Group 3 from the control animals serve as links in the plasma that are reflective of mechanisms in the liver, as revealed in the correlation analyses in the later sections.

### Correlation Networks for Finding Plasma Biomarkers Reflective of Drug-Induced Liver Toxicity Mechanisms

As can be seen from the above analyses, in the plasma of this mammalian model many hundreds of biomolecules were measured whose abundances are statistically significantly altered due to the administration of the toxic compound to this mammalian system. However, previous studies have shown that not every such disregulated biomolecule is disregulated due to a direct consequence of toxicity. Indeed, many if not most of the observed changes can be due to ancillary or secondary effects to the drug-induced toxicological insult, such as acute phase responses, appetite changes due overall poor health, and the like.

The primary objective of the study was to select, among all measured changes in the plasma of drug-administered animals, biomarkers of hepatic steatotic processes (changes in analytes due to ancillary or secondary effects are not of interest in this study as they presumably do not comprise direct information reflective of and relevant to the molecular toxicological processes in the liver).

One large correlation network was created from all the analytes measured in both liver and plasma. Urine to liver correlations were calculated, but because none of these correlations passed the established threshold criterion, urine peaks were excluded from the final correlation network. The criteria used to generate the correlation networks resulted in a network with 210 plasma nodes and 3570 liver nodes. This correlation network is not entirely graphically connected—there are many disjointed sub-graphs within the network, but one very large sub-graph encompasses nearly all of the nodes in the correlation network.

### Correlation Network Details.

| | FDR nodes | Correlation | | |
|---|---|---|---|---|
| Compartment | threshold^{a} | threshold^{b} | Nodes | Edges^{c} |
| Plasma | 0.15 | 0.75 | 210 | 172 |
| Liver | 0.15 | 0.8^{d} | 3570 | 17327 |

^{a}The maximum FDR p value allowed for a node to be called significantly different between any of the groups.
^{b}The minimum absolute value of the correlation between a node in the specified compartment with a node in the liver for the edge to be included in the network.
^{c}The number of edges between nodes in the specified compartment and liver nodes.
^{d}In addition, this correlation threshold had to satisfy an FDR p-value less than 0.15.

### Selection Criteria for Correlation Sub-Networks

As the entire correlation network is very large, a strategy was devised for selecting portions of the network for further investigation. Because one focus of the study was to identify plasma biomarkers of liver toxicity, sub-networks were generated by selecting liver nodes that are correlated to plasma nodes (“hubs”) and included all nodes that are connected by a correlation edge to this liver node. These sub-networks can be disjoint or joint. This methodology captures the plasma nodes that correlate to liver hub nodes and also all other liver nodes that are correlated to the hub in a single sub-network. To focus on the sub-networks that are most likely of greatest interest, sub-networks were generated only where the hub exhibited a statistically significant change in expression when comparing Group 3 with Group 1 and also where the hub was correlated to other liver nodes. This selection process generated many sub-networks, however only one will be discussed below for illustration.

The plasma-to-liver correlation network was built with partial correlations which are robust across analysis of all groups. Partial correlations were calculated instead of correlations within a particular group because limited numbers of animals were used in the study. This method involves calculating correlations after group specific means are removed, which allows one to discount spurious associations between two analytes that can appear due to differences in expression levels between treatment groups in either one or both of the analytes considered. Although these correlations are valid irrespective of the drug dose, the sub-networks are relevant to illustrating the effects of toxicity because the plasma nodes and the hub liver node exhibit statistically significant changes in the comparison between the high drug dose and control groups (Group 3 and Group 1, respectively).

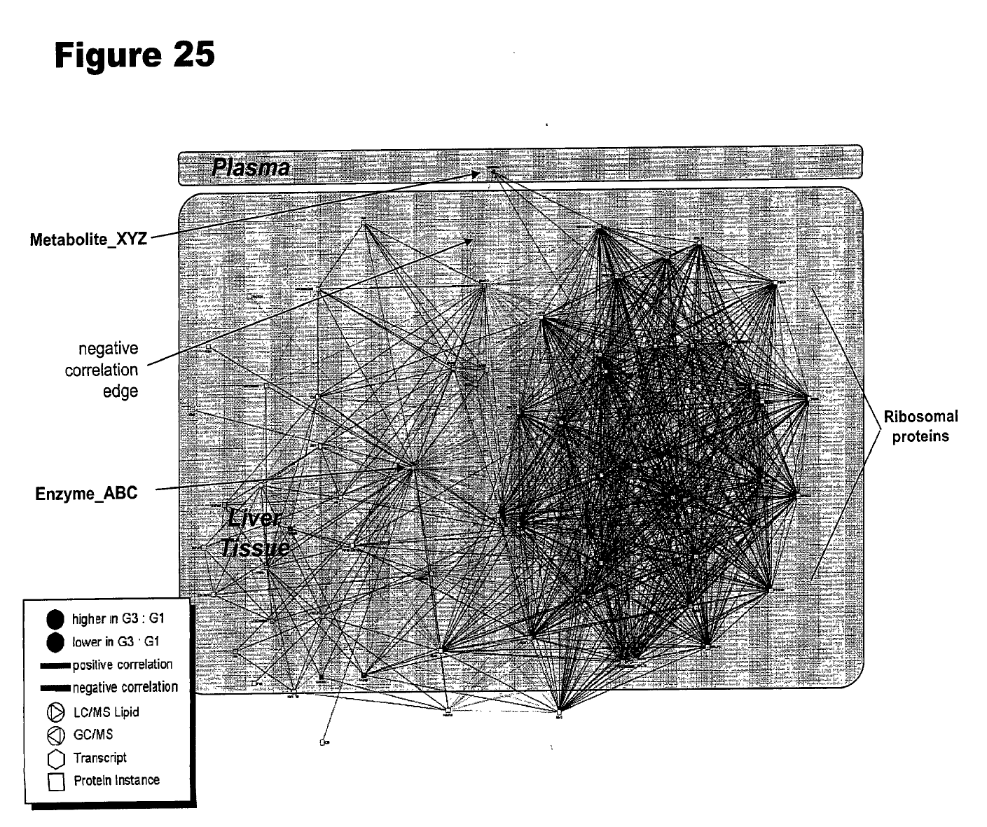

FIG. 25 shows one of the selected correlation sub-networks. In this network, Enzyme_ABC, which is reduced in abundance in the liver tissue of the Group 3 drug-administered animals relative to the Group 1 control animals (by approximately 2-fold as measured by the proteomics platform, and by approximately 1.7-fold as measured by the mRNA transcript platform) was calculated to be negatively correlated with circulating Metabolite_XYZ in plasma (Metabolite_XYZ is increased in abundance by approximately 1.4-fold in the Group 3 drug-administered animals compared to Group 1 animals as measured by the plasma GC-MS platform).

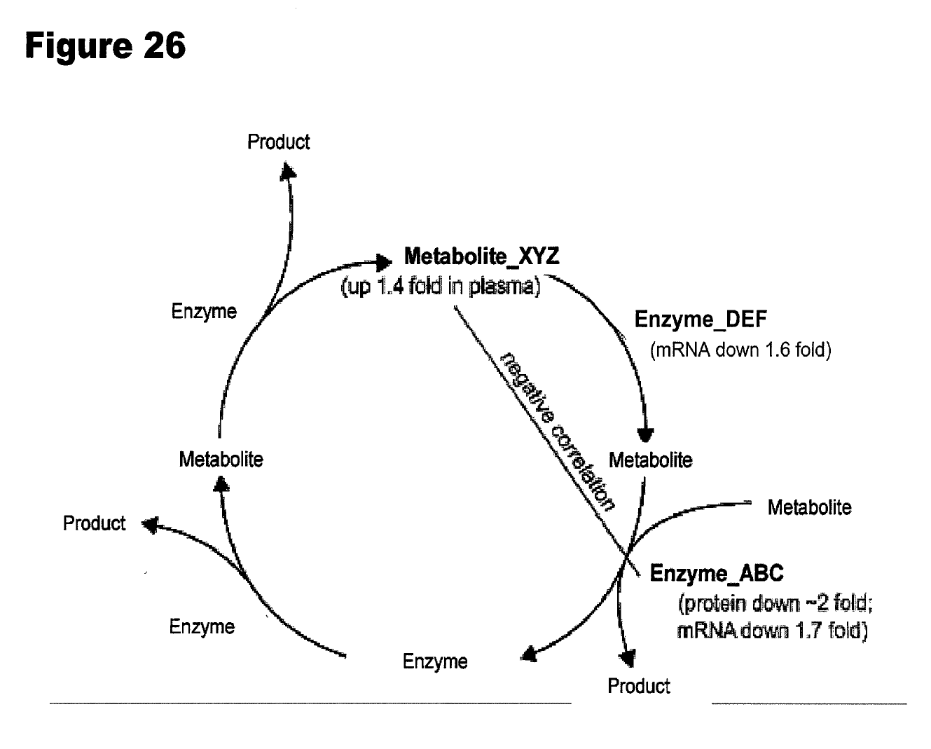

FIG. 26 illustrates graphically a hypothesis as to the biochemical situation which may give rise to this observation. In FIG. 26, a biochemical cycle is shown in which both Enzyme_ABC and Metabolite_XYZ are known to play a role. It is striking to see a negative correlation between Enzyme_ABC and plasma Metabolite_XYZ, which is upstream of Enzyme_ABC in this cycle. In addition, the mRNA transcript for Enzyme_DEF, an enzyme downstream of Enzyme_ABC in this cycle, was measured to be lower in abundance in Group 3 compared to Group 1. Metabolite_XYZ is not a direct substrate of Enzyme_ABC, but is indeed a known substrate of Enzyme_DEF, the enzyme that precedes Enzyme_ABC in this biochemical cycle and which produces the product which is the substrate for Enzyme_ABC. A plausible hypothesis is therefore that the administration of the toxic drug deregulates this biochemical cycle by decreasing the abundance of Enzyme_DEF, breaking the cycle at that point and leading to an accumulation of Metabolite_XYZ and a reduction of Enzyme_ABC. The more Enzyme_ABC is reduced, the more the greater the accumulation of Metabolite_XYZ.

Through prior research, it has been reported that juvenile visceral steatosis mice have lower levels of enzymes related to this biochemical cycle in the liver; these mice have a defect in a compound involved in transferring fatty acids, which results in both steatosis and lower levels of all cycle enzymes. It has also been shown that long-chain fatty acids can suppress induction of genes encoding enzymes of this biochemical cycle. Taken together, these studies indicate that there may be a link between fatty acid metabolism and this biochemical cycle that is observable through monitoring of plasma biomarkers such as Metabolite_XYZ.

As such, it is hypothesized by these measurements in liver tissue and plasma of mRNA, proteins and metabolites that this excess Metabolite_XYZ finds its way into the plasma. Therefore, plasma levels of Metabolite_XYZ is postulated to be a specific and sensitive, and easily accessible and observable, biomarker for the disruption of this biochemical cycle by the hepatotoxicological effects of this drug compound.

In addition, it is seen from FIG. 25 that Metabolite_XYZ in plasma is positively correlated to a number of ribosomal proteins in the liver tissue. The significance of these correlations have not yet been explored.

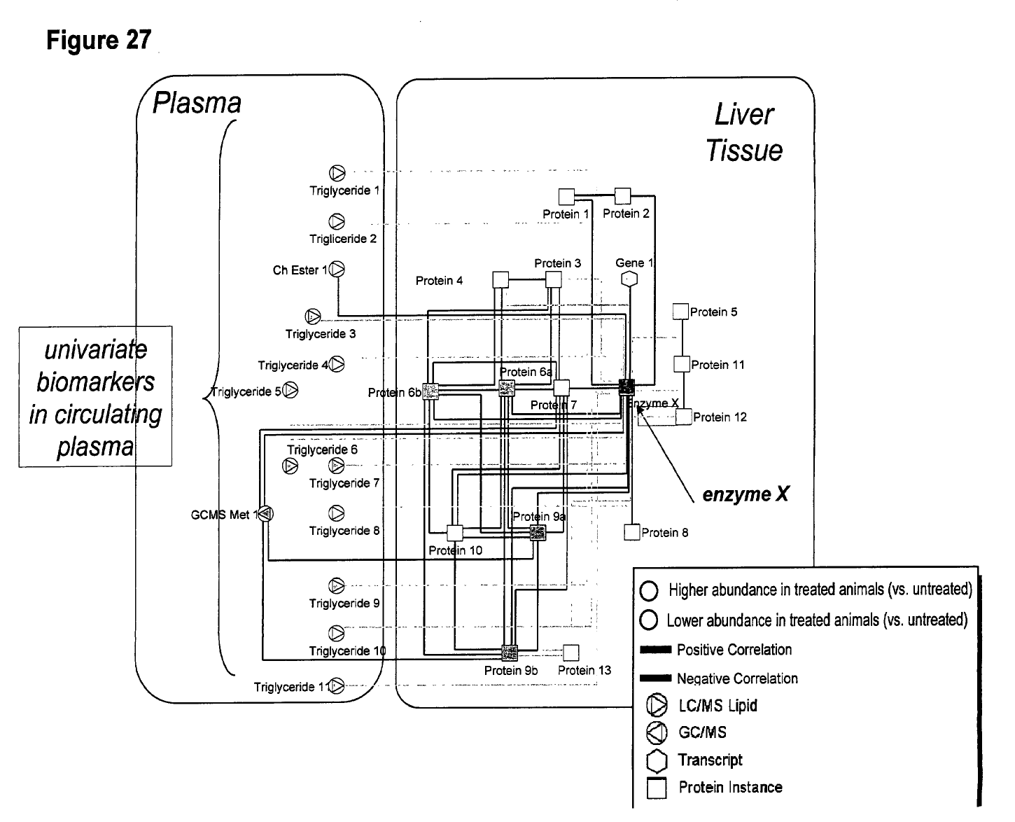

Another plasma-to-liver correlation network which was found to be persistent across all treatment groups was centered upon hepatic Enzyme_X and a number of plasma lipids, as shown in FIG. 27. This correlation network was pursued because of the established role of Enzyme_X in hepatic drug metabolism processes, and as such this network most likely reflects liver exposure to the compound. FIG. 27 is a visualization of the correlation matrix centered upon hepatic Enzyme_X, illustrating significant correlations both to other liver analytes as well as to analytes observed in plasma. In FIG. 27, each observed protein, gene transcript and endogenous metabolite is assigned a node co-ordinate in the two-dimensional plane, and the links between nodes represent correlation values between pairs of nodes. The network in FIG. 27 has been constrained to comprise only analytes which are separated by one correlation link from Enzyme_X; apart from this constraint this correlation analysis is unsupervised.

In conclusion, one primary challenge of molecular toxicology is to discern between changes in abundances of biomolecular analytes which are due to direct toxicological phenomena and effects which are due to ancillary or secondary phenomena. From the table, it can be seen that the plasma GC-MS analytical platform selected many hundreds of plasma features which were statistically significantly disregulated upon drug administration in this animal system. However, a systems-wide integrative correlation approach has selected and prioritized one measurement from this platform, namely Metabolite_XYZ, as a key biomarker directly reflective of a hepatic steatosis-involved biochemical process. This finding is direct information reflective of and relevant to the molecular toxicological processes in the liver associated with the toxicity of the drug under study.

Variations, modifications, and other implementations of what is described herein will occur to those of ordinary skill in the art without departing from the spirit and the essential characteristics of the present teachings. Accordingly, the scope of the present teachings is to be defined not by the preceding illustrative description but instead by the following claims, and all changes that come within the meaning and range of equivalency of the claims are intended to be embraced herein.

1. A correlation analysis data set characteristic of a biological state of an animal,
the data set recorded in retrievable form comprising a plurality of derived values indicative of a measure of the degree of correlation between groups of data points representative of the relative concentrations of a multiplicity of biomolecules detectable in a sample from an animal in a biological state, the values together serving to characterize said biological state and being distinct from a set of values derived in the same way in a comparable sample from an animal of the same species in a different biological state.

2. The correlation analysis data set of claim 1 wherein the values indicative of a measure of the degree of correlation between groups of data points comprise correlation coefficients indicative of the degree of correlation between multiple pairs of said data points.

3. A correlation map comprising the correlation analysis data set of claim 1 displayed in a format permitting visual identification of the biological state of the animal.

4. A set of correlation analysis data sets of claim 1 comprising plural different said correlation analysis data sets or correlation maps, respectively, representative of different biological states in the same or different animals.

5. The set of claim 4 wherein the plural different correlation analysis data sets or correlation maps are representative of the biological state of an animal at different time points, or are representative of the biological state of an animal treated with different drugs or different drug doses, or include a data set representative of the biological state of an animal and a data set representative of the biological state of a human.

6. The correlation analysis data set of claim 1 wherein the derived values comprise at least two different types of measurements of a sample of a biological system.

7. The correlation analysis data set of claim 1 wherein the derived values are preprocessed derived values.

8. A set of correlation analysis data sets of claim 1 comprising a test animal correlation data set and a reference data set comprising values derived from multiple samples taken from a plurality of animals known to be in said biological state.

9. A set of correlation analysis data sets of claim 1 comprising plural different said correlation analysis data sets or correlation maps, respectively, representative of different biological compartments in the same or different animals.

10.-12. (canceled)

13. The correlation analysis data set of claim 1 wherein the biomolecules comprise at least two of proteins, peptides, nucleic acids, lipids, and metabolites.

14. The correlation analysis data set of claim 1 wherein the animal is a human or an experimental animal.

15. The correlation analysis data set of claim 1 wherein the biological state is a pathologic, diseased, well, toxic, homeostatic, hunger-induced, environmentally-induced, exercise-induced, drug-induced, placebo-induced, or mental illness-induced state.

16. The correlation analysis data set of claim 1 wherein the biomolecules comprise proteins, peptides, nucleic acids, lipids, and metabolites.

17.-21. (canceled)

22. A systems biology analysis method comprising:
determining values indicative of negative or positive correlations between or among the levels of plural biomolecules present in biological samples from one or more animals in one or more preselected biological states; and
selecting plural values indicative of correlations among said biomolecules for inclusion in a data set comprising a profile characteristic of said one or more biological states.

23. The method of claim 22 comprising assessing the clustering coefficient of biomolecules included in said data set to determine a biomolecule for consideration as a target to modulate said biological state.

24. The method of claim 22 comprising displaying at least a portion of the data set to produce a correlation map characteristic of a biological state.

25. The method of claim 22 wherein plural different data sets are selected to respectively representative different biological states of the same or different animals.

26. The method of claim 23 wherein the plural different data sets are representative of the biological state of an animal at different time points, or are representative of the biological state of an animal treated with different drugs or different drug doses, or include a data set representative of the biological state of an animal and a data set representative of the biological state of a human.

27. (canceled)

28. The method of claim 22 wherein said sample is whole blood, a blood fraction, urine, saliva, lymph, cerebrospinal fluid, a liquefied tissue sample, mucous, nipple secretion, feces, ocular fluid, or a combination thereof.

29. The method of claim 22 wherein the biological state is a pathologic, diseased, well, toxic, homeostatic, hunger-induced, environmentally-induced, exercise-induced, drug-induced, placebo-induced, or mental illness-induced state.

30. The method of claim 22 wherein the biomolecules comprise at least two of proteins, peptides, nucleic acids, lipids, and metabolites.

31.-33. (canceled)

34. A method for assessing the efficacy of a drug candidate for treating a disease state, said method comprising:
a) providing a test correlation analysis data set characteristic of a biological state of an animal, the test correlation analysis data set comprising a plurality of derived values indicative of a measure of the degree of correlation between groups of data points representative of the relative concentrations of a multiplicity of biomolecules detectable in a sample from an animal to which a drug candidate has been administered, the derived values together serving to characterize the drugged state;
b) providing a reference correlation analysis data set comprising a corresponding plurality of derived values indicative of a measure of the degree of correlation between groups of data points representative of the relative concentrations of the same multiplicity of biomolecules detectable in a sample from a different individual or multiple individuals of the same species as said animal to which the drug candidate has not been administered and which do not exhibit the disease state or have been effectively treated for the disease state; and
c) comparing the test correlation analysis data set and reference correlation analysis data set, a substantial similarity of the test correlation analysis data set with the reference correlation analysis data set being indicative of probable efficacy.

35.-37. (canceled)

38. A method for assessing the toxicity of a substance, the method comprising the steps of:
a) providing a test correlation analysis data set characteristic of a toxic state of an animal comprising a plurality of derived values indicative of a measure of the degree of correlation between groups of data points representative of the relative concentrations of a multiplicity of biomolecules detectable in a sample from a said animal to which the substance has been administered, the derived values together serving to characterize the toxic state;
b) providing a reference correlation analysis data set comprising said plurality of derived values indicative of a measure of the degree of correlation between groups of data points representative of the relative concentrations of the same multiplicity of biomolecules detectable in a sample from a said animal to which the substance has not been administered, the sample(s) used to generate the reference data set being obtained from a different individual of the same species as the first animal, multiple animals of the same species as the first animal, the same animal, or a different animal, and
c) comparing the test correlation analysis data set with the reference correlation analysis data set.

39. The method of claim 38 wherein the sample used to generate the reference correlation analysis data set is obtained from an animal to which a known toxin has been administered, and a substantial similarity of the test correlation analysis data set with the reference correlation analysis data set is indicative of probable toxicity.

40. A method for assessing the toxicity of a substance, the method comprising:
a) providing a test correlation analysis data set characteristic of a toxic state of an animal comprising a plurality of derived values indicative of a measure of the degree of correlation between groups of data points representative of the relative concentrations of a multiplicity of biomolecules detectable in a sample from an animal to which the substance has been administered, the derived values together serving to characterize the toxic state;
b) providing a reference correlation analysis data set comprising a plurality of derived values indicative of a measure of the degree of correlation between groups of data points representative of the relative concentrations of the same multiplicity of biomolecules detectable in a sample from a different individual or multiple individuals of the same species as said animal, which individuals have not been exposed to or administered the substance, and which have been treated with a different substance known to be toxic to animals of said species, and
c) comparing the test and reference correlation analysis data sets.

41.-51. (canceled)
